(19) United States
(12) Patent Application Publication
(10) Pub. No.: US 2014/0072662 A1
(43) Pub. Date: Mar. 13, 2014

(54) Andrographis Paniculata Compositions and Methods for Treatment of Addictions

(71) Applicant: Omeros Corporation, Seattle, WA (US)

(72) Inventor: Roberto Ciccocioppo, Camerino (IT)

(21) Appl. No.: 13/934,659

(22) Filed: Jul. 3, 2013

Related U.S. Application Data
(60) Provisional application No. 61/668,909, filed on Jul. 6, 2012.

(51) Int. Cl. A61K 36/19; A61K 36/738; A61K 36/185; A61K 31/192; A61K 31/352; A61K 36/8888; A61K 31/12; A61K 31/045; A61K 31/365; A61K 36/40; A61K 36/8884; A61K 36/234; A61K 36/48; A61K 31/202

(52) U.S. Cl. CPC A61K 36/19; A61K 36/48; A61K 36/738; A61K 36/185; A61K 31/192; A61K 31/352; A61K 31/202; A61K 31/12; A61K 31/045; A61K 31/365; A61K 36/40; A61K 36/8884; A61K 36/234; A61K 36/8888
USPC 424/757; 424/725; 424/765; 424/776

(57) ABSTRACT

Methods and compositions for the treatment or prevention of addictions and impulse control disorders using Andrographis paniculata, active substances contained therein and extracts thereof, alone or in combination with other therapeutic agents. The methods and compositions of the invention are useful in the treatment or prevention of addiction to substances, including alcohol, nicotine, marijuana, opioid agonists cocaine, and amphetamines, as well as impulse control disorders, including pathological gambling and pathological overeating.

### CROSS-REFERENCE TO RELATED APPLICATION

This application claims the benefit under 35 U.S.C. §119(e) of U.S. Provisional Patent Application No. 61/668,909 filed Jul. 6, 2012, and the specification of this provisional application is incorporated herein by reference in its entirety.

### FIELD OF THE INVENTION

The present invention is directed generally to the treatment or prevention of addictions and impulse control disorders using Andrographis paniculata, active substances contained therein and extracts thereof, alone or in combination with other therapeutic agents.

### BACKGROUND OF THE INVENTION

Drug addiction is a chronic relapsing disorder, characterized by compulsion and craving to seek and take the drug, loss of control over drug intake, and emergence of negative emotional states when access to the drug is discontinued (Koob and Le Moal, 1997, 2008). Essentially, the path leading to addiction follows a course of social drug-taking, related with the hedonic effects of the drug (positive reinforcement) and known as drug recreational use. By increasing dosages, the recreational use may lead to perceiving the “need” or “craving” for the drug that in turn may move in a pattern of escalating compulsive use. This transition ultimately drives to a dependence state, where the continued drug use is rather aimed to prevent the withdrawal syndrome (negative reinforcement).

The World Health Organization (WHO) defines substance addiction as using a substance repeatedly, despite knowing and experiencing harmful effects. Substance addiction is a chronic, relapsing disease characterized by a loss of control over drug use, compulsive drug seeking and craving for a substance, use that persists despite negative consequences, and physical and/or psychological dependence on the substance. Substance addiction typically follows a course of tolerance, withdrawal, compulsive drug taking behavior, drug seeking behavior, and relapse. Substance abuse and addiction are public health issues with significant social and economic impact on both the addict and society by playing a major role in violent crime and the spread of infectious diseases. Addictive substances include alcohol, caffeine, nicotine, cannabis (marijuana) and cannabis derivatives, opiates and other morphine-like opioid agonists such as heroin, phencyclidine and phencyclidine-like compounds, sedative ipnotics such as benzodiazepines and barbiturates and psychostimulants such as cocaine, amphetamines and amphetamine-related drugs such as dextroamphetamine and methylamphetamine.

Alcohol is one of the most commonly abused substances at a global level. Particularly in Europe, about 58 million adults (16%) are classified as heavy drinkers, of which about 23 million (6%) are alcoholics. Europeans spend ˜ [symbol] 100 billion on alcoholic beverages annually, which is reflected by the high rate of alcohol consumption per capita of 10 litres of pure alcohol per year. Similarly, the alcohol consumption in North America in the last decade averaged 8.5 litres per year (Spanagel, 2009). Excessive alcohol drinking is a condition that in the United States affects more than 12% of the population at some point in their life (Hasin et al, 2007). Consuming and abusing these huge amounts of alcohol clearly drives to detrimental consequences with enormous socio-economic and health impacts on the world population. Elevated alcohol consumption is in fact associated with costly, adverse social consequences, such as disruption of families, crime, traumatic accidents, and lost productivity. Additionally, alcoholism leads to serious liver and cardiovascular disease and generates dependence resulting in severe mental disorders, social problems and adverse consequences including the division of families, tragic accidents and the reduction of work performance. According to the WHO, alcohol consumption is responsible for 20-30% of oesophageal and liver cancer, liver cirrhosis, homicides, epilepsy, and motor vehicle accidents worldwide. Globally, alcohol abuse leads to about 1.8 million deaths per year. According to the European Status Report on Alcohol and Health 2010, the total tangible cost of alcohol to the European Union was estimated to be [symbol] 125 billion, equivalent to 1.3% of the gross domestic product. Actual spending on alcohol-related problems accounts for [symbol] 66 billion of this, while potential production not realized due to absenteeism, unemployment and premature mortality accounts for a further [symbol] 59 billion.

Compulsive behaviour towards the consumption of alcohol is a core symptom of the disorder. In recent years several approaches have been investigated to help alcoholic patients to not only control alcohol drinking but also alcohol cravings and relapse (Monti et al., 1993; Volpicelli et al. 1992; O'Brien et al. 1997). Medications such as naltrexone, acamprosate, ondansetron, disulfuram, gamma hydroxybutyrate (GHB), and topiramate have been tested for their potential therapeutic effect on alcohol abuse belong to several classes (Volpicelli et al. 1992; O'Brien et al. 1997). Few of these pharmacotherapeutics, such as naltrexone, acamprosate, and disulfuram, have been proven to be of a certain utility and approved for the treatment of alcoholism. Among these medications, the non-selective opioid antagonist naltrexone is currently considered the pharmacological gold standard. However, despite some promising results none of these medications, including naltrexone, is of sufficient efficacy in alcoholism and prognosis remains poor.

Currently available pharmacotherapies for alcohol addiction are only moderately successful, continue to be viewed with considerable scepticism outside the scientific community and have not become widely adopted as treatments (Heilig et al, 2011). Indeed, the clinical diagnosis of alcoholism follows well established criteria as reported in the Diagnostic and Statistical Manual of Mental Disorders [currently in its fourth edition, (DSM IV), see table 1], contributing to eliminate some of the subjective judgment involved in making diagnoses. However, approximately three-quarters of those in the general population who meet diagnostic criteria for alcoholism never receive treatment (Hasin et al, 2007). Therefore, new therapeutic approaches for treatment of alcoholism are needed.

Nicotine is one of the most widely used addictive drugs, and nicotine abuse is the most common form of substance abuse. The WHO estimates that there are 1.25 billion smokers worldwide, representing one third of the global population over the age of 15. The WHO further estimates that 5 million deaths occur each year as a direct result of tobacco use, making nicotine abuse the largest single preventable cause of death worldwide. In industrialized countries, 70-90% of lung cancer, 56-80% of chronic respiratory disease, and 22% of cardiovascular disease instances are attributed to nicotine addiction. Cigarette smoking is associated with 430,000 deaths a year in the US alone and is estimated to cost the nation 80 billion dollars yearly in health care costs. Tobacco use accounts for one third of all cancers, including cancer of the lung, mouth, pharynx, larynx, esophagus, cervix, kidney, ureter, and bladder. The overall rates of death from cancer are twice as high among smokers as among nonsmokers. Smoking also causes lung diseases such as chronic bronchitis and emphysema; exacerbates asthma symptoms; and increases the risk of heart disease, including stroke, heart attack, vascular disease, and aneurysm. An estimated 20% of the deaths from heart disease are attributable to smoking. Expectant women who smoke are at greater risk than nonsmokers for premature delivery, spontaneous abortion, and infants with decreased birth weight.

Nicotine use results in increased levels of the neurotransmitter dopamine, which activates the reward pathways to regulate feelings of pleasure and to mediate the desire to consume nicotine. Symptoms associated with nicotine withdrawal include craving, irritability, anger, hostility, aggression, fatigue, depression, and cognitive impairment, which lead the abuser to seek more nicotine. Environmental conditioning factors and exposure to psychological stress represent additional factors motivating nicotine use in smokers. Repeated nicotine use results in the development of tolerance, requiring higher doses of nicotine to produce the same initial stimulation.

Most therapies developed for nicotine addiction have shown only moderate success in preventing relapse, leading to a high failure rate in attempts to quit smoking Treatments include the use of nicotine replacement products, antidepressants, antihypersensitives, and behavioral therapy.

The National Institute on Drug Abuse estimates that 72 million Americans, about one third of the population, have tried marijuana. Acute effects of marijuana use include memory and learning problems, distorted perception, difficulty problem solving, loss of coordination, and increased heart rate. Long term abuse can cause the same respiratory problems observed in tobacco smokers, such as daily cough, phlegm production, increased risk of lung infections, and an increased chance of developing cancer of the head, neck and lungs. Depression, anxiety, and job-related problems have been associated with marijuana use. Long term marijuana use can result in addiction with compulsive use that interferes with daily activities. Cravings and withdrawal symptoms, such as irritability, increased aggression, sleeplessness, and anxiety make it difficult for addicts to stop using marijuana. There are no pharmaceutical treatments available for treating marijuana addiction and relapse.

According to the WHO, an estimated 13 million people abuse opiates worldwide, including 9 million heroin addicts. More than 25% of opiate abusers die from suicide, homicide, or an infectious disease, such as HIV and hepatitis, within 10-20 years of becoming addicted. Tolerance and physical dependence can develop within two to three days. While abuse and addiction to opioid agents is a known phenomenon, there has been a worsening of this problem in recent years (Compton and Volkow 2006; Compton and Volkow 2006). Epidemiological surveys of youth in the United States in 2003 indicated that opioid analgesics were among the most frequently abused illicit drugs among secondary students (12th graders), second only to marijuana (Delva et al. 2005). Furthermore, the past few years have seen a marked increase in the use of opioid medications in the United States and an even greater increase in problems associated with such use. This upsurge in use and problems is particularly concerning because it seems to represent an expanded pathway to opioid addiction (Siegal, Carlson et al. 2003).

According to recent epidemiological data, 4.7% (i.e., 11.0 million) United States household residents over the age of twelve abused an opioid medication in 2002, and 13.7% of these people (i.e., 1.5 million) exhibited the symptoms of a DSM-IV opioid use disorder (Association 1994; Substance Abuse and Mental Health Services Administration 2004). As recently reviewed by Compton and Volkow, the annual incidence of opioid analgesic abuse increased from 628,000 initiates in 1990 to 2.4 million initiates in 2001 (Administration 2003; Substance Abuse and Mental Health Services Administration 2003). One of the reasons behind the expansion of opioid addiction is the increased use of analgesic secondary to medical prescription. Short term use of opioid medication is rarely associated with addiction. Conversely, protracted treatments with these agents have been associated with development of addiction in up to 18% of patients.

The goals for treatment of opiate addiction, as with other types of substance addictions, are to discontinue the use of the opiate while minimizing painful withdrawal symptoms and preventing relapse. Current treatments involve replacing the addictive drug with a substitution of an opioid receptor agonist or mixed agonist/antagonist. An alternative approach consists of the use of an opioid receptor antagonist to block the effect of the agonist. Antagonists provide no relief from pain or other withdrawal symptoms; rather, they can precipitate withdrawal, and their therapeutic use was associated with increased accidental opioid agonists overdosing and increased lethality. Use of agonists with a lower affinity for the receptors results in the least severe withdrawal symptoms, but it can lead to a dependence on the substitute opiate. Also, many substitution therapies take 3-6 months, allowing time for addicts to stop treatment midway through the course of treatment.

Psychostimulants, such as cocaine and amphetamines, cause euphoria, increased alertness, and increased physical capacity in humans. These substances first increase dopamine transmission, but long term drug usage results in a reduction of dopamine activity, leading to dysregulation of the brain reward system and dysporia. The WHO estimates 33 million people around the world abuse amphetamines.

Chronic cocaine abuse can result in hyperstimulation, tachycardia, hypertension, mydriasis, muscle twitching, sleeplessness, extreme nervousness, hallucinations, paranoia, aggressive behavior, and depression. Cocaine overdose may lead to tremors, convulsions, delirium, and death resulting from heart arrhythmias and cardiovascular failure. Desipramine, amantadine and bromocriptine have been shown to decrease cocaine withdrawal symptoms.

Amphetamine withdrawal symptoms include EEG changes, fatigue, and mental depression. Tolerance develops over time and may be associated with tachycardia, auditory and visual hallucinations, delusions, anxiety reactions, paranoid psychosis, exhaustion, confusion, memory loss, and prolonged depression with suicidal tendencies. Current treatments for amphetamine addiction include phenothiazines, haloperidol, and chlorpromazine for hallucinations, but potential side effects of these drugs include postural hypotension and severe extrapyramidal motor disorders.

In the past, treatment for substance addictions focused on behavioural therapy, but dependence on many of these highly addictive substances is hard to break. In particular, addictions to alcohol, cocaine, and heroin are considered chronic, relapsing disorders. Also, concurrent abuse of multiple substances, such as nicotine, heroin, cocaine and alcohol, is common.

The associated medical, social and occupational difficulties that develop during the course of addiction do not disappear after detoxification and changes in brain due to addictive drugs endure long after the patient stops taking them, accounting for high risks of relapse (O'Brien and McLellan, 1996). Clinical evidence indicates that 40% to 60% of patients treated for drug dependence return to active substance use within a year following treatment discharge (McLellan et al, 2000).

The long-lasting, chronic nature of many addictions and high rates of recidivism present a considerable challenge for the treatment of drug and alcohol addiction, such that understanding of the neurobiological basis of relapse has emerged as a central issue in addiction research. Emotional and environmental factors (conditioning stimuli) were listed among the main causes of relapse. For example, it is known that specific stress conditions such as loss of work and economic difficulties, or stimuli predictive of the presence of alcohol previously associated with its use, such as a bottle of the preferred wine and a bar-like environment, may strongly facilitate relapse in detoxified former alcoholics.

Clearly, there is a need in the art for new methods for treating and preventing addiction and the relapse use of addictive agents. The present invention meets these needs by providing methods and nutraceutical compositions useful in treating and preventing addiction, including addiction to addictive substances and practice of behaviours associated with impulse control disorders, as well as reducing relapse use of addictive substances and relapse practice of behaviours associated with impulse control disorders.

### SUMMARY OF THE INVENTION

The present invention is directed to methods of treating substance addiction or an impulse control disorder or reducing the likelihood of relapse use of an addictive substance or practice of a behavior associated with an impulse control disorder by administering to a subject in need thereof a composition including an effective amount of Andrographis paniculata or an active substance or extract of Andrographis paniculata, in which the composition is effective to treat the substance addiction or impulse control disorder or reduce the likelihood of relapse use of the addictive substance or practice of the behavior associated with the impulse control disorder.

In one aspect of the invention, the composition includes an extract of Andrographis paniculata in a solvent. In another aspect of the invention, the composition includes dehydroandrographolide, andrographolide or neoandrographolide.

In another aspect of the invention, the composition includes an effective amount of Andrographis paniculata or an active substance or extract of Andrographis paniculata in a nutraceutical base. In another aspect of the invention, the composition further comprises one or more polyunsaturated fatty acids that upregulate peroxisome proliferator-activated receptor gamma (PPARγ), such as polyunsaturated fatty acid selected from eicosapentaenoic acid (EPA), conjugated linoleic acid (CLA) and docosahexaenoic acid (DHA), and in a further aspect includes the Omega-3 fatty acid combination of EPA, CLA and DHA.

In another aspect of the invention, the Andrographis composition includes one or more additional botanicals, or active substances contained therein or extracts thereof, that upregulate peroxisome proliferator-activated receptor gamma (PPARγ), such as rose oil, citronellol and/or geraniol, pomegranant seed oil, abscisic acid, punicic acid, red clover extract, genistein, Biochanin A, curcumin, Cornus kousa, Cistus Salvifolius, Glycyrrhiza glabra roots, Albizia julibrissin, Arisaema sp., Cnidium monnieri, Pinellia ternata and Tribulus terrestris.

A further aspect of the present invention is directed to methods of treating substance addiction or an impulse control disorder or reducing the likelihood of relapse use of an addictive substance or practice of a behavior associated with an impulse control disorder by administering to a subject in need thereof a composition including an effective amount of a botanical selected from rose oil, citronellol and/or geraniol, pomegranant seed oil, abscisic acid, punicic acid, red clover extract, genistein, Biochanin A, curcumin, Cornus kousa, Cistus Salvifolius, Glycyrrhiza glabra roots, Albizia julibrissin, Arisaema sp., Cnidium monnieri, Pinellia ternata and Tribulus terrestris, or an active substance or extract thereof, in which the composition is effective to treat the substance addiction or impulse control disorder or reduce the likelihood of relapse use of the addictive substance or practice of the behavior associated with the impulse control disorder.

The methods and compositions of the present invention may be used to treat addiction of an addictive substance, such as alcohol, nicotine, marijuana, a marijuana derivative, an opioid agonist, a benzodiazepine, a barbiturate, or a psychostimulant. The methods and compositions of the present invention may also be used to treat an impulse control disorder, such as pathological gambling, pathological overeating, pathological use of electronic devices, pathological use of electronic video games, pathological use of electronic communication devices, pathological use of cellular telephones, addiction to pornography, sex addiction, obsessive compulsive disorder, compulsive spending, binge eating disorder, food addiction, anorexia, bulimia, intermittent explosive disorder, kleptomania, pyromania, trichotillomania, compulsive overexercising, and compulsive overworking.

In another aspect of the present invention, the compositions of the present invention use Andrographis paniculata or an active substance or extract of Andrographis paniculata in combination with one or more additional therapeutic agents, for treating substance addiction or an impulse control disorder or reducing the likelihood of relapse use of an addictive substance or practice of a behavior associated with an impulse control disorder, wherein each of the Andrographis paniculata or an active substance or extract of Andrographis paniculata and the additional therapeutic agent contribute to the effective treatment or reduction of relapse practice of the addiction. Accordingly, the present invention provides methods and related compositions, unit dosage forms, and kits useful for the treatment and prevention of addictions, and for the treatment and prevention of relapse use of addictive agents or practice of addictive or compulsive behaviors.

In one embodiment, the present invention includes a method of treating or preventing an addiction, comprising determining that a subject has or is at risk of developing an addiction, and providing to the subject an amount of Andrographis paniculata or an active substance or extract of Andrographis paniculata effective for the treatment or prevention of the addiction.

In certain embodiments, the additional therapeutic agent is an opioid antagonist, a mixed opioid partial agonist/antagonist, an antidepressant, an antiepileptic, an antiemetic, a corticotrophin-releasing factor-1 (CRF-1) receptor antagonist, a selective serotonin-3 (5-HT3) antagonist, a 5-HT_{2A/2C} antagonist, or a cannabinoid-1 (CB1) receptor antagonist. In particular embodiments, the opioid antagonist is naltrexone or nalmefene. In particular embodiments, the antidepressant is fluoxetine, mirtazapine, or bupropion. In particular embodiments, the antiepileptic is topiramate, levetiracetam, or gabapentin. In one embodiment, the CRF-1 receptor antagonist is antalarmin. In another embodiment, the selective serotonin-3 (5-HT3) antagonist is ondansetron. In particular embodiments, the cannabinoid-1 (CB1) receptor antagonist is rimonabant or tanarabant. In one embodiment, the mixed opioid agonist/antagonist is buprenorphine.

In another aspect of the present invention, the compositions of the present invention include Andrographis paniculata or an active substance or extract of Andrographis paniculata in combination with one or more additional addictive agents, wherein the Andrographis paniculata or an active substance or extract of Andrographis paniculata reduces the likelihood that a subject will develop an addiction to the addictive agent or reduces the likelihood that the subject will relapse to addictive use of the addictive agent. In one embodiment of the invention, the addictive substance may be nicotine or an opioid agonist. In a related aspect of the invention, related compositions, unit dosage forms, and kits including the addictive agent and Andrographis paniculata or an active substance or extract of Andrographis paniculata are provided.

### BRIEF DESCRIPTION OF THE DRAWINGS

FIG. 1 provides the chemical structures of active substances included in Andrographis paniculata.

FIG. 2 illustrates the effect of A. paniculata (AP) on alcohol intake in msP rats. Voluntary 10% alcohol intake levels (g/kg) following per OS (oral) treatment with A. paniculata (0, 15 and 150 mg/kg) administered 12 hours and 1 hour before access to alcohol. Data were recorded at 0.5 (a), 2 (b), 8 (c) and 24 (d) hours after the availability of alcohol. Data represent the mean±S.E.M. Significance was *p<0.05 vs. Veh (vehicle); where not indicated, differences from vehicle were not statistically significant.

FIG. 3 illustrates the effect of A. paniculata (AP) on alcohol intake in msP rats. Voluntary 10% alcohol intake levels (g/kg) following per OS treatment with A. paniculata (0 and 450 mg/kg) administered 12 hours and 1 hour before access to alcohol. Data were recorded at 0.5 (a), 2 (b), 8 (c) and 24 (d) hours after the availability of alcohol. Data represent the mean±S.E.M. Significance was*p<0.05 **,p<0.01, ***p<0.001 vs. Veh (vehicle); where not indicated, difference from vehicles were not statistically significant.

FIG. 4 illustrates the effect of A. paniculata (AP) on yohimbine-induced reinstatement of Alcohol Seeking in msP rats (N=8). During training phase animals reached a stable baseline of 10% (v/v) alcohol solution responding. Extinction sessions progressively decreased the number of responses. Compared to extinction (EXT), yohimbine (1.25 mg/kg, i.p.) elicited a significant reinstatement of responding that was affected by A. paniculata pre-treatment (0, 150 and 450 mg/kg, i.p.). Values represent the mean (SEM) number of responses at (a) alcohol active lever and (b) inactive lever. Significant difference from extinction was found and noted as ###p<0.001. Significant difference from vehicle (Veh) was found and noted as ***p<0.001.

FIG. 5 illustrates the effect of A. paniculata (AP) on cue-induced reinstatement of alcohol seeking in msP rats (N=9). The first bar represents extinction: the mean number of lever presses (a, active; b, inactive) of the last 3 days of extinction (Ext). The next four bars represent the results of the reinstatement test: responses in rats treated with A. paniculata (0, 150 and 450 mg/kg) and exposed to both S⁺/CS⁺ and S⁻/CS⁻ conditions (in the absence of reward delivery). Conditioned reinstatement of alcohol seeking was not modified by treatment with A. paniculata under both S⁺/CS⁺ and S⁻/CS⁻ conditions. The significance of the difference from extinction was ##p<0.01; for the difference from vehicle-treated rats (Veh), p>0.05.

FIG. 6 illustrates the effect of andrographolide (AND) on alcohol intake in msP rats, showing voluntary 10% alcohol intake levels (g/kg) following i.p. treatment with andrographolide (5 and 10 mg/kg) and vehicle administered 12 hours and 30 minutes before access to alcohol. Data were recorded at 0.5, 2, 8 and 24 hours after the availability of alcohol. Data represent the mean±S.E.M. Significance was *p<0.05, **p<0.01 vs vehicle. Where not indicated, differences from vehicle were not statistically significant.

FIG. 7 illustrates the effect of andrographolide (AND) on food intake in msP rats, showing voluntary food intake levels (g/kg) following i.p. treatment with AND (5 and 10 mg/kg) and vehicle administered 12 hours and 30 minutes before access to alcohol. Data were recorded at 0.5, 2, 8 and 24 hours after the availability of food. The data represents the mean±S.E.M. Differences from vehicle were not statistically significant.

FIG. 8 illustrates voluntary 10% alcohol intake following chronic intracerebroventricular injection of 5 μg/rat of the PPAR antagonist GW9662 (GW 5) and of 10 mg/kg of andrographolide (AND) at 0.5, 2, 8 and 24 hours. The data represents the mean SEM±. Differences from vehicle treated group had statistical significance of *p<0.05, **p<0.01 and ***p<0.001. Differences from andrographolide treated group had significance of #p<0.05, ##p<0.01 and ###p<0.001. Where not indicated, differences from vehicle were not statistically significant.

FIG. 9 illustrates voluntary food intake levels following chronic intracerebroventricular injection of 5 μg/rat of the PPAR antagonist GW9662 (GW5) and of 10 mg/kg of andrographolide (AND) at 0.5, 2, 8 and 24 hours. The data represents the mean SEM±. Where not indicated, differences from vehicle were not statistically significant.

FIG. 10 illustrates the effect of A. paniculata (AP) on yohimbine-induced reinstatement of Nicotine Seeking in Wistar rats (N=15) that are trained to self-administration of nicotine; then, during extinction responses, the active lever activates the delivery mechanism but does not result in the delivery of nicotine. The day after the last extinction session, in a within subject counterbalanced design animals are given Andrographis extract OS at the doses of 0, 150; 450 mg/kg given 1 hour prior to administration of yohimbine (1.25 mg/kg) or its vehicle. 30 minutes following the administration of yohimbine, animals are placed in the self-administration apparatus, and lever pressesis recorded for 2 hours. In a counterbalance Latin square design, each animal receives all drug treatments. A 2-day interval, during which animals are subjected to extinction sessions, occurs between each drug test. One way ANOVA revealed a significant reinstatement of responding in yohimbine treated rats compared to extinction (p<0.001).

### DETAILED DESCRIPTION

The present invention is directed to nutraceutical compositions including Andrographis paniculata or an active substance or extract of Andrographis paniculata as a natural substance for use in the treatment of addictions, including substance addiction and behaviors associated with impulse control disorders, and for the prevention or reduction of relapse use of an addictive agent or behavior.

### A. DEFINITIONS

As used herein, unless the context makes clear otherwise, “treat,” and similar word such as “treatment,” “treating” etc., is an approach for obtaining beneficial or desired results, including and preferably clinical results. Treatment can involve optionally either the reducing or amelioration of a disease or condition, (e.g., addiction, relapse use, withdrawal), or the delaying of the progression of the disease or condition (e.g., addiction relapse use, withdrawal).

As used herein, unless the context makes clear otherwise, “prevent,” and similar word such as “prevention,” “preventing” etc., is an approach for preventing the onset or recurrence of a disease or condition, (e.g., addiction, relapse use, withdrawal) or preventing the occurrence or recurrence of the symptoms of a disease or condition, or optionally an approach for delaying the onset or recurrence of a disease or condition or delaying the occurrence or recurrence of the symptoms of a disease or condition. Preventing also includes inhibiting the onset or recurrence of a disease or condition, or one or more symptoms thereof, and reducing the likelihood of onset or recurrence of a disease or condition, or one or more symptoms thereof.

“Andrographis paniculata”, “A. paniculata” or “AP” refer to the herb or botanical also commonly known as chiorta, creat, green chirayta, king of bitters, chiretta, chuan xin lian, kalamegha and kirayat.

An “active substance” of Andrographis paniculata refers to a pharmacologically active compound contained in Andrographis paniculata, and includes dehydroandrographolide, andrographolide (also referred to herein as “AND”) and neoandrographolide. While not wishing to be limited by theory, the present inventor believes that these active substances act by mediating peroxisome proliferator-activated receptor gamma (PPARγ), such as by upregulating or acting functionally as an agonist of PPARγ.

An “extract” of Andrographis paniculata refers to a composition including one or more active substances of Andrographis paniculata that has been extracted from Andrographis paniculata using a solvent, such as water, ethanol or an edible oil, in either its solution form or as a solute dried from such a solution.

As used herein, the term “Andrographis” refers to Andrographis paniculata, an active substance of Andrographis paniculata or an extract of Andrographis paniculata.

Generally, a subject is provided with an effective amount of Andrographis. As used herein, an “effective amount” or a “therapeutically effective amount” of a substance, e.g., Andrographis, is that amount sufficient to affect a desired biological or psychological effect, such as beneficial results, including clinical results. For example, in the context of treating addiction using the methods of the present invention, an effective amount of Andrographis is that amount sufficient to cause the subject to reduce or discontinue use of an addictive agent.

### B. ANDROGRAPHIS PANICULATA AND ADDICTION

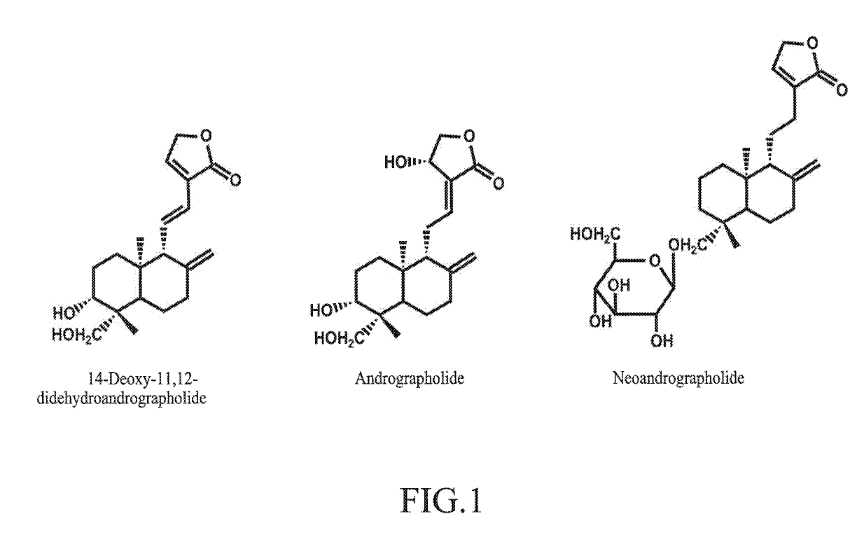

Andrographis paniculata (Burm. f) Nees (A. paniculata), literally ‘king of bitters’ is a herbaceous plant belonging to the Family Acanthaceae, used in traditional Siddha and Ayurvedic medicine as well as in tribal medicine in India and some other countries for multiple clinical applications, such as rheumatoid arthritis and inflammatory symptoms of sinusitis (Coon and Ernst, 2004; Deng, 1985). A. paniculata possesses potent antiinflammatory activity and contains several compounds, including dehydroandrographolide, andrographolide and neoandrographolide (Koteswara Rao et al, 2004; Parichatikanond et al, 2010; Reddy et al, 2003). Among these compounds, andrographolide is considered to be the most active and important constituent of this plant. FIG. 1 provides the structures of dehydroandrographolide, andrographolide and neoandrographolide.

Andrographolide has been reported to have multiple pharmacological properties, such as antipyretic (Suebsasana et al, 2009), antiinflammatory (Shen et al, 2002), antiallergic (Xia et al, 2004), antiplatelet aggregation (Amroyan et al, 1999), antiviral (Wiart et al, 2005), anti-HIV (Reddy et al, 2005), antithrombotic (Thisoda et al, 2006), and, similarly to thiazolidinediones (TDZs) such as pioglitazone, antidiabetic activities (Reyes-Balaguer et al, 2005; Yu et al, 2008). It also shows immunostimulatory (Xu et al, 2007), hepatoprotective (Amroyan et al, 1999), and anticancer activities by inhibition of cell cycle progression (Shi et al, 2008). Importantly, a clinical study demonstrated ability of andrographolide to cross the brain-blood barrier (Lu, 1995) while exhibiting clear neuroprotective effects in a rat model of cerebral ischemia (Chan et al, 2010). Also, andrographolide and rosiglitazone were shown to share antiinflammatory properties (Iruretagoyena et al, 2006). Furthermore, evidence exists that andrographolide exerts its antiinflammatory properties through a PPARγ-mediated mechanism (Hanke Orozco et al, 2006). In accordance with the present invention, the inventor investigated the effects of A. paniculata and its active substance andrographolide on alcohol consumption and seeking in rats.

The inventor has demonstrated in accordance with the present invention, as detailed herein below, that a subchronic treatment with A. paniculata significantly reduces voluntary alcohol intake and yohimbine-induced, but not cue-induced reinstatement of alcohol seeking in rats genetically selected for high alcohol consumption. The reduced alcohol consumption was also observed by the present inventor when using the major and the most active constituent of A. paniculata, andrographolide. The effect was mediated by PPARγ because it was prevented by pretreatment with a PPARγ antagonist, GW9662.

As described in greater detail in the examples below, the reduction of voluntary alcohol intake observed following the oral treatment with A. paniculata at 15 and 150 mg/kg dose occurred during the 4 days of treatment, with drug efficacy increasing from the first administration day. The effect of A. paniculata was even more pronounced following administration of 450 mg/kg. Alcohol consumption returned to baseline levels following drug discontinuation. At low drug doses (15-150 mg/kg), the reduction of alcohol intake was only observed at 0.5-hours from extract administration. Drug effect was longer (up to 24 hrs) following administration of a higher dose (450 mg/kg). Overall, these data indicate that active substances (i.e., andrographolide) contained in the A. paniculata are rapidly absorbed into the blood following oral administration and quickly metabolized. On the other hand it is also known that, due to its low aqueous solubility, andrographolide has a low oral bioavailability and high binding to plasma proteins. For this reason, its bioavailability has been estimated to decrease four-fold when a 10-times-higher dose was used (Panossian et al, 2000). Therefore, pharmacokinetic properties of constituents of A. paniculata may account for both the observed lack of dose-response correlation and the short-term pharmacological effect. On the other hand, at the highest dose tested (450 mg/kg) the effect on voluntary alcohol consumption was substantially longer lasting suggesting the fact that these pharmacokinetic limitations could be at least in part overcome by increasing the dosage of plant extract.

As a main active ingredient of A. paniculata, and without being limited by theory, andrographolide is thought by the inventor to be responsible for the pharmacological actions of the plant. Thus, the inventor evaluated the effect of andrographolide on voluntary alcohol intake under identical conditions of those used by the inventor to examine the effects of A. paniculata. Results obtained clearly confirmed those observed with A. paniculata, with andrographolide being able to reduce alcohol consumption at oral doses of 5 and 10 mg/kg dose. Resembling data obtained with the A. paniculata extract, the effect progressively increased over repeated administrations. While alcohol intake returned to baseline levels after drug treatment was discontinued. Water and food intake were not affected by administration of andrographolide, despite a slight trend to an increased food intake was observed at 0.5 hours. This data suggests the specificity of the andrographolide effect on alcohol intake, which cannot be attributed to a general inhibition of feeding behavior.

To demonstrate that andrographolide-induced decrease of alcohol consumption was mediated by PPARγ, the inventor used the selective PPARγ antagonist GW9662 (5 μg/rat) and demonstrated if this antagonist is given ICV prior to andrographolide (10 mg/kg, OS), it completely prevents the andrographolide effect on alcohol drinking.

Alcoholism is defined as a chronic relapsing disease. Therefore, studies on alcohol addiction are aimed not only at identifying remedies suitable to decrease alcohol consumption but also for alcohol seeking prevention. Activation of the brain stress system is thought to play a pivotal role in alcohol addiction, and stress is a major factor triggering relapse in abstinent alcoholics (Koob, 2008). Yohimbine, an α-2 adrenoceptor antagonist, increases cell firing and release of brain noradrenalin, and acts as a pharmacologic stressor (Abercrombie et al, 1988; Aghajanian and VanderMaelen, 1982; Holmberg et al, 1962; Le et al, 2005; Lee et al, 2004). Yohimbine is known to increase anxiety-like symptoms related to alcohol abuse in humans (Charney et al, 1983; Umhau et al, 2011) and in rats, and importantly this drug has been shown to reinstate alcohol seeking following extinction in rats trained to self-administer alcohol (Le et al, 2005; Marinelli et al, 2007). This pharmacological stressor was, therefore, used by the inventor to investigate the effect of A. paniculata on reinstatement of alcohol seeking behavior, as further described in the examples below.

Results demonstrated that pretreatment with A. paniculata at both doses of 150 and 450 mg/kg significantly reduced relapse-like behavior induced by yohimbine. A. paniculata was in fact able to return responding at the previously alcohol-paired lever to extinction levels. Neither yohimbine nor A. paniculata had effects on inactive lever responding, providing evidence of action specificity. Environmental conditioning factors are also known to play a pivotal role in alcohol relapse (Ludwig and Wikler, 1974a; Ludwig et al, 1974b; McCusker and Brown, 1990; Monti et al, 1993).

To evaluate the effect of A. paniculata on cue-induced relapse-like behavior, the inventor trained rats on an extinction/reinstatement procedure in which re-exposure to cues predictive of alcohol availability elicited a robust resumption of lever responding (Ciccocioppo et al, 2001; Ciccocioppo et al, 2001b). As detailed below, A. paniculata did not show any activity in this model, indicating a high degree of specificity to inhibit stress-induced relapse-like behavior while relapse induced by environmental stimuli was not altered. This observation strongly suggests the involvement of A. paniculata on stress-induced alcohol-related processes. Evidence that stress interacts with alcohol-induced neuroadaptations, that may drive core symptoms of alcoholism, including the increased motivation to take alcohol (Breese et al, 2005a) strengthens these results.

In addition to the classical neurobiological substrates associated with stress and alcohol vulnerability such as GABA, serotonin, norepinephrine, and corticotrophin-releasing factor (Aston-Jones and Kalivas, 2008; Breese et al, 2005b; Koob, 2010), recent data also implicate the cytokine/chemokine systems in central nervous system sensitization (Kawasaki et al, 2008) as well as in the etiology of chronic alcohol-related phenotypes (Breese et al, 2008).

It is known that alcohol, possibly through involvement of toll-like receptor 4 (TLR4) (Alfonso-Loeches et al; Dolganiuc et al, 2006; Rostene et al, 2007; Szabo et al, 2007), is able to release cytokines that in turn alter neural activity at brain sites involved in the regulation of anxiety-like behaviors (Bajetto et al, 2002; Kreutzberg, 1996). For example, both lipopolysaccharide (LPS) toxin insult or administration of cytokines/chemokines accelerate the development of anxiety-like behavior in animals subsequently exposed to alcohol and subjected to withdrawal (Breese et al, 2008). Similarly, profound enhancement of LPS effects on brain cytokines has been observed in rodents pretreated with chronic alcohol (Qin et al, 2008). Further, elevated levels of the chemokine monocyte chemotractant protein-1 (MCP-1/CCL2) has been found in several regions of the human post-mortem alcoholic brain including the amygdala (He and Crews, 2008). Moreover, intra central amygdala (CeA) microinjection of the cytokine TNFα facilitated alcohol withdrawal-induced anxiety, suggesting that an extra-hypothalamic action of cytokines contributes to chronic alcohol effects on behavior (Knapp et al, 2011). Consistent with cytokines having a role in mediating emotional responses (Adler et al, 2005; Bauer and Patterson, 2006; Rostene et al, 2007; Shi et al) a link between the immune system and depression, stress and anxiety has been also established, (Anisman et al, 2003; Dunn et al, 2005; Hayley et al, 2005; Pucak and Kaplin, 2005; Raison et al, 2006; Uddin et al).

Interestingly, previous work from the inventor's laboratory showed strong effects from activation of PPARγ with pioglitazone on alcohol consumption, withdrawal and seeking behavior (Stopponi et al, 2010). Glial function was hypothesized to underlying these effects. To strengthen this view, further evidence showed that glia-derived neurotrophic factor reduced cocaine abuse vulnerability (Messer et al, 2000) and lowered alcohol consumption (He et al, 2005), while partial deletion of the glial cell-derived neurotrophic factor transcript increased sensitivity to morphine and methamphetamine reward (Airavaara et al, 2007; Yan et al, 2007). Moreover, activation of glia-derived proinflammatory mediators such as interleukin (IL)-1β, IL-6, and tumor necrosis factor (TNF)-α increased locomotor and neurotoxic effects of psychostimulants (Ladenheim et al, 2000; Zalcman et al, 1999) and at least in part mediated the development of opiate tolerance (Lin et al; Mika, 2008; Tsai et al, 2008). Finally, a recent study described that administration of the TNF-α antagonist, etanercept, caused large reductions in rapid-eye movement sleep in abstinent alcoholic patients (Irwin et al, 2009), suggesting a role of this sleep abnormalities to increase risk of relapse vulnerability (Brower, 2003).

Consistent with the inventor's hypothesis that the effects of A. paniculata on alcohol abuse depends on its ability to modulate the neuroimmune response via activation of PPARγ receptors, it has been shown that the major active constituent of the plant, andrographolide, inhibits the production of TNF-α, IL-1β and IL-12 in LPS-stimulated macrophages (Abu-Ghefreh et al, 2009; Qin et al, 2006). Further, current evidence suggests that andrographolide attenuates inflammation through the inhibition of NF-kB, an important transcription factor responsible for the inflammatory response (Chan et al, 2010; Iruretagoyena et al, 2006; Wang et al, 2007; Xia et al, 2004), that in turn attenuates cytokine release (Bao et al, 2009) and LPS-induced microglial activation (Wang et al, 2004). Altogether, and while not wishing to be limited by theory, this evidence led the inventor to conclude that A. paniculata effects on drinking are mediated by its neuroimmune modulatory properties.

### C. ADDICTIVE SUBSTANCES AND IMPULSE CONTROL DISORDERS

The term addiction is used to describe a recurring compulsion by an individual to engage in some specific activity, despite harmful consequences to the individual's health, mental state or social life. The term “addiction” is often reserved for substance addictions, but as used herein with respect to the compositions and treatments of the present invention also refers to other compulsions, such as problem gambling, compulsive overeating and other impulse control disorders. Factors that have been suggested as causes of addiction include genetic, biological/pharmacological and social factors.

The medical community now makes a careful theoretical distinction between physical or physiological dependence (characterized by symptoms of withdrawal) and psychological dependence (sometimes referred to simply as addiction). Addiction is now narrowly defined as “uncontrolled, compulsive use.” If there is no harm being suffered by, or damage done to, the patient or another party, then clinically it may be considered compulsive, but to the definition of some it is not categorized as “addiction”. In practice, the two kinds of addiction (physiological dependence and psychological dependence) are not always easy to distinguish. Addictions often have both physical and psychological components.

Physical dependence (or drug dependence) refers to a state resulting from habitual use of a drug, where negative physical withdrawal symptoms result from abrupt discontinuation. Examples of addictive agents for which a user may develop a physical dependence include nicotine, opioids, barbiturates, benzodiazepines, alcohol, i.e., ethyl alcohol, GHB, and methaqualone.

Commonly abused stimulants such as cocaine or amphetamine class drugs are not believed to cause significant physical dependence. However, their potential for extreme physiological addiction can compel the user to consume amounts which become physically damaging, but life-threatening withdrawal effects have not been observed.

As used herein with respect to the disorders to be treated by the methods and compositions of the present invention, addictive agents includes any and all agents to which a subject can become addicted, either physically or psychologically, or both. As noted above, addictions to be treated by the methods and compositions of the present invention include addiction to chemical entities, such as drugs, e.g., ethyl alcohol, nicotine, or cocaine, as well as addiction to other behaviors, e.g., pathological gambling, pathological overeating, pathological use of electronic devices, e.g., smart phones, pathological use of electronic video games, pathological use of electronic communication devices, pathological use of cellular telephones, addiction to pornography, sex addiction, obsessive compulsive disorder, compulsive spending, binge eating disorder, food addiction, anorexia, bulimia, intermittent explosive disorder, kleptomania, pyromania, trichotillomania, compulsive overexercising, and compulsive overworking.

Addiction to addictive agents to be treated with the methods and compositions of the present invention include addictive recreational drugs, as well as addictive medications. Examples of addictive agents include, but are not limited to, alcohol, e.g., ethyl alcohol, gamma hydroxybutyrate (GHB), caffeine, nicotine, cannabis (marijuana) and cannabis derivatives, opiates and other morphine-like opioid agonists such as heroin, phencyclidine and phencyclidine-like compounds, sedative ipnotics such as benzodiazepines, methaqualone, mecloqualone, etaqualone and barbiturates and psychostimulants such as cocaine, amphetamines and amphetamine-related drugs such as dextroamphetamine and methylamphetamine. Other examples include LSD, psilocybin, ecstasy and other hallucinogens. Examples of addictive medications include, e.g., benzodiazepines, barbiturates, and pain medications including alfentanil, allylprodine, alphaprodine, anileridine benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofenitanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, OXYCONTIN®, oxymorphone, papaveretum, pentazocine, phenadoxone, phenomorphan, phenazocine, phenoperidine, piminodine, piritramide, propheptazine, promedol, properidine, propiram, propoxyphene sufentanil, tramadol, tilidine, salts thereof, mixtures of any of the foregoing, mixed μ-agonists/antagonists, and the like.

In certain embodiments of the present invention, a subject may be addicted to an opioid agonist. The terms “opioid agonist,” “opioid” and “opiate” are used interchangably herein and are used to designate a group of drugs that are, to varying degrees, opium- or morphine-like in their properties. Their main use is for pain relief. These agents work by binding to opioid receptors, which are found principally in the central nervous system and the gastrointestinal tract. Opiates are also addictive agents. Opiates include, e.g., alfentanil, allylprodine, alphaprodine, anileridine, apomorphine, benzylmorphine, beta-hydroxy 3-methylfentanyl, bezitramide, carfentanil, clonitazene, codeine, desomorphine, dextromoramide, diacetylmorphine (heroin), diampromide, dihydrocodeine, dihydroetorphine, dihydromorphine, dimenoxadol, dimepheptanol, dimethylthiambutene, dioxaphetylbutyrate, dipipanone, eptazocine, ethoheptazine, ethylmethylthiambutene, ethylmorphine, etonitazene, etorphine, fentanyl, hydrocodone, hydromorphone, hydroxypethidine, isomethadone, ketobemidone, LMM, levorphanol, Ievophenacylmorphan, lofentanil, meperidine, metapon, metazocine, methadone, methadyl acetate, metopon, morphine, myrophine, narceine, nicomorphine, norlevorphanol, normethadone, normorphine, norpipanone, opium, oxycodone, oxymorphone, papaverine, phenadoxone, phenomorphan, phenoperidine, piminodine, piritramide, propheptazine, promedol, properidine, propoxyphene, remifentanil, sufentanil, thebaine, tildine, and tramadol.

Naturally occurring opiates include, e.g., codeine, morphine, noscapine, papaverine, and thebaine. Semi-synthetic opioids include, e.g., diacetylmorphine, hydrocodone, hydromorphone, levorphanol, metapon, nalorphine, naloxone, naltrexone, oxycodone, oxymorphone, and tramadol. Synthetic opioids include, e.g., ethoheptazine, fentanyl, levorphanol, meperidine, methadone, phenazocine, propoxyphene and sufentanil.

Three broad classifications of opiates are phenanthrenes, phenylheptylamines, and phenylpiperidines. Examples of phenanthrenes include codeine, etorpine, hydrocodone, hydromorphone, morphine, oxycodone, and oxymorphone. Examples of phenylheptylamines include dimeheptanol, dimenoxadol, dipipanone, isomethadone, methadone, methadyl acetate, and propoxyphene. Examples of phenylpiperidines include alfentanyl, alphaprodine, beta-promedol, carfentanyl, fentanyl, lofentanil, meperidine, properidine, and sufentanil.

In certain embodiments of the present invention, a subject may be addicted to a psychostimulant. Specific psychostimulants include, by way of example, amphetamine, cocaine, dextroamphetamine, methamphetamine, pemoline, and methylenedioxymethamphetamine.

While a subject may be addicted to a single addictive agent or behavior, frequently, a subject is addicted to two or more addictive agents or behaviors. Addiction to two or more addictive agents or addictive behaviors is referred to as polyaddiction, and may also be treated in accordance with the present invention.

### D. METHODS OF TREATING ADDICTIONS AND REDUCING RELAPSE TO ADDICTION USING ANDROGRAPHIS

The present invention includes methods of treating or preventing an addiction, comprising providing an effective amount of Andrographis (Andrographis paniculata or an active substance, such as andrographolide, or extract of Andrographis paniculata) to a subject having an addiction or at risk for developing an addiction. In various embodiments, the subject is addicted to an addictive agent or behavior, including, but not limited to, any of the addictive agents and behaviors described herein. The subject may be physically or physiologically dependent on the substance or behavior; the subject may be psychologically dependent; or the subject may be both physically and psychologically dependent. The subject may be addicted to one or more than one addictive agent or behavior.

According to certain embodiments of the present invention, a subject is provided with Andrographis, while in other embodiments, a subject is provided with Andrographis in combination with an additional therapeutic agent. It is understood that the effective amount of either or both of Andrographis and an additional therapeutic agent may be different when either is provided alone than when provided in combination. For example, when Andrographis and the additional therapeutic agent act synergistically, then a lower amount of the Andrographis, a lower amount of the additional therapeutic agent, or lower amounts of both the Andrographis or the additional therapeutic agent may be required to achieve the same therapeutic effect that would be provided by either the Andrographis or the additional therapeutic agent alone. In other embodiments, the same amount of the Andrographis and the additional therapeutic agent are used to provide an enhanced therapeutic effect relative to the therapeutic effect provided by either the Andrographis or the additional therapeutic agent alone.

The subject treated in accordance with the present invention may be any animal, including a mammal, and, particularly, a human.

In one aspect of the invention, the subject is first determined or diagnosed to have an addiction, or to be at risk of developing an addiction, by diagnostic testing, observation or analysis by a medical care provider. An effective amount of Andrographis, or an effective amount of Andrographis and one additional therapeutic agent, are then provided to the subject for treatment or prevention of the addiction. In another aspect of the invention, the subject is first determined or diagnosed to have an addiction, or to be at risk of developing an addiction, by diagnostic testing, observation or analysis by a medical care provider, but the subject has not been diagnosed or determined to have diabetes or other insulin disorder. An effective amount of Andrographis, or an effective amount of Andrographis and an additional therapeutic agent, are then provided to the subject for treatment or prevention of the addiction. The dosage of Andrographis, Andrographis and an additional therapeutic agent, may be specifically determined by the medical practitioner for treatment or prevention of the addiction rather than for any other disorder or disease.

In particular aspects, the subject is provided with Andrographis, alone or in combination with an additional therapeutic agent for the primary purpose of treating or preventing an addiction. In related aspects of the methods of the present invention, the subject has not previously been provided with Andrographis for the treatment or prevention of any disease or disorder other than an addiction. In particular, in certain embodiments, the subject has not previously been provided with Andrographis for the treatment of insulin resistance or diabetes. In a further related embodiment, the subject has not been diagnosed with insulin resistance or diabetes.

In particular embodiments, the subject is suffering from or at risk for addiction to any physically addictive agent or addictive or compulsive behavior, including, e.g., any of those described below. In particular embodiments, the subject is addicted to alcohol, cocaine, nicotine, marijuana, an opiate or other opioid agonist or methamphetamine or other psychostimulant, or phencyclidine and phencyclidine derivatives.

In particular embodiments, a subject is considered at risk of addiction or relapse to use of an addictive agent or practice of an addictive or compulsive behavior when the subject has previously been addicted to the same or a different addictive agent or addictive or compulsive behavior. In certain embodiment, the subject is considered at risk of addiction or relapse to use of an addictive agent or practice of an addictive or compulsive behavior when the subject is psychologically addicted to an addictive agent or addictive or compulsive behavior, even if the subject is no longer physically addicted.

In certain embodiments, the subject is addicted to or at risk of becoming addicted to a therapeutic agent provided to the patient to treat a disease or disorder, e.g., a pain medication. In a related embodiment, the subject may be at risk of abusing an addictive therapeutic agent, such as a pain medication. Abusing an addictive therapeutic agent, in certain embodiments, is understood to indicate using the agent for a reason different than or in addition to its prescribed use. In such a situation, a subject may be provided with both an addictive therapeutic agent and Andrographis, alone or in combination with an additional therapeutic agent. For example, a subject suffering from pain, or at risk of pain, may be provided with an opioid agonist and Andrographis, to both provide analgesia and prevent or treat addiction to the opioid agonist. Because Andrographis is believed to have antiinflammatory properties, Andrographis may add to or enhance the analgesic effect of the opioid agonist.

In various embodiments, the subject is provided with Andrographis at the same time that the subject is using an addictive agent, after the subject has discontinued use of an addictive agent, or before the subject begins using an addictive agent. In particular embodiments, Andrographis and the addictive agent (e.g., nicotine, an opioid agonist or alcohol) agonist may be provided in a single co-formulation or composition. For example, a subject being treated with a controlled dosage of nicotine for purposes of eliminating or reducing the use of tobacco products may be Andrographis at the same time, in order to increase the likelihood that the subject will reduce or cease use of the tobacco product. In particular embodiments, Andrographis and nicotine may be provided in a single co-formulation or composition.

### E. METHODS OF TREATING ADDICTION AND REDUCING RELAPSE TO ADDICTION USING ANDROGRAPHIS IN COMBINATION WITH OTHER THERAPEUTIC AGENTS

In accordance with the present invention, Andrographis may be effectively used in combination with one or more additional therapeutic agents to treat or prevent addiction, including addiction to one or more of the addictive agents described infra and compulsive or addictive behavior. Accordingly, the present invention includes methods of treating or preventing an addiction, comprising providing to a subject addicted to, or at risk of becoming addicted to, an addictive agent or suffering from an impulse control disorder Andrographis and one or more additional therapeutic agent(s), in which each of the Andrographis and the additional therapeutic agent(s) contribute to the effective treatment or prevention of the addiction. In some embodiments, the additional agent is another antiaddiction agent.

Andrographis and the additional therapeutic agent may be administered at the same time (i.e., concurrently), or either may be administered before the other (i.e., sequentially). In general, both Andrographis and the additional therapeutic agent are present in the subject at the same time for a duration of time and at levels sufficient to provide a therapeutic benefit to the subject, i.e., in the treatment or preventing of an addiction or the prevention of a relapse use (or reinstatement) of an addictive agent or compulsive or addictive behavior. The Andrographis and the additional therapeutic agent may be administered by the same or different routes of administration. Typically, the Andrographis and the additional therapeutic agent are each provided to a subject according to a standard route of administration of a commercially available or other pharmaceutical composition. In one embodiment, Andrographis and the additional therapeutic agent are co-administered using a composition comprising both agents.

The additional therapeutic agent provided in combination Andrographis may be any therapeutic agent that contributes to an aspect of the effective treatment or prevention of the addiction. For example, the additional therapeutic agent may be a drug used to treat an addiction or a drug used to alleviate side-effects associated with physiological withdrawal from an addictive agent. In addition, the additional therapeutic agent may be any drug that affects brain serotonin neurotransmission, such as selective serotonin reuptake inhibitors (SSRIs), and tricyclic and tetracyclic serotonin and norepinephrine reuptake inhibitors (SNRIs) as described below, and serotonin agonists such as sumatriptan, ergonovine, dihydroergotamine and buspirone. In certain embodiments, the additional therapeutic agent is an opioid antagonist, including mixed opioid partial agonist/antagonists, an antidepressant, an antiepileptic, an antiemetic, a corticotrophin-releasing factor-1 (CRF-1) receptor antagonist, a selective serotonin-3 (5-HT3) antagonist, a 5-HT_{2A/2C} antagonist such as mianserin, mirtazapine and ketanserin, or a cannabinoid-1 (CB1) receptor antagonist, including but not limited to those therapeutic agents specifically described infra.

In one aspect of the invention, the additional therapeutic agent administered with Andrographis is one or more polyunsaturated fatty acids that upregulate peroxisome proliferator-activated receptor gamma (PPARγ), such as polyunsaturated fatty acid selected from eicosapentaenoic acid (EPA), conjugated linoleic acid (CLA) and docosahexaenoic acid (DHA). In a further aspect includes the Omega-3 fatty acid combination of EPA, CLA and DHA, which Omega-3 fatty acids may be contained in or derived from a plant source, such as flax seeds, soy bean products or walnuts, or an animal source, e.g., fish oil or krill oil.

In another aspect of the invention, the additional therapeutic agent administered with Andrographis is one or more botanical agents, or active substances contained therein or extracts thereof, that upregulate peroxisome proliferator-activated receptor gamma (PPARγ), such as rose oil, citronellol and/or geraniol, pomegranant seed oil, abscisic acid, punicic acid, red clover extract, genistein, Biochanin A, curcumin, Cornus kousa, Cistus Salvifolius, Glycyrrhiza glabra roots, Albizia julibrissin, Arisaema sp., Cnidium monnieri, Pinellia ternata and Tribulus terrestris.

Rose oil is an essential oil that is widely used in perfumes and cosmetics, and has been reported to possess a wide range of physiologic activities including analgesic, hypnotic and anti-inflammatory properties. (Katsukawa et al. (2011)). Major components of rose oil include citronellol, geraniol and nerol. Citronellol and geraniol, but not nerol, have been demonstrated to activate PPARγ (Katsukawa et al. (2011)). Therefore, in accordance with the present invention, rose oil, citronellol and geraniol are believed to be useful in accordance with the present invention, used in combination with Andrographis or used alone, to treat addiction or to prevent relapse to practice of an addiction.

Bassaganya-Riera at al. (2011) summarized the effects of three plant-derived PPAR agonists: abscisic acid (ABA), punicic acid (PUA) and catalpic acid (CAA) in the prevention and treatment of chronic inflammatory and metabolic diseases and disorders. In plants, the isoprenid phytohormone abscisic acid is involved in numerous developmental and stress responses, and has been determined to increase the activity of PPARγ in 3T3-L1 preadipocytes. [Bassaganya-Riera at al (2011)]. Conjugated linolenic acids (CLnAs) or conjugated triene fatty acids are found as triglycerides in the seed oils of some plants belonging to the Punicaceae, Bignoniaceae, Rosaceae, Curcubitaceae, and Euphorbiaceae families. Glycerides from these plant sources provide an easily accessible source of these unusual types of fatty acids, including but not limited to punicic (PUA), jacaric acid (JAA) catalpic (CAA), and eleostearic acids (ESA); all of which have demonstrated some promising health effects by acting as dual or panagonists of PPARs, as summarized in Bassaganya-Riera at al (2011). PUA also known as trichosanic acid is a conjugated triene fatty acid naturally found at high concentrations in the seed of Punica granatum (Punicaceae, Pomegranate) and Trichosanthes kirilowii, and constitutes 64-83 percent of the pomegranate seed oil (PSO). Bassaganya-Riera recently demonstrated a dose-dependent increase in the ability of PUA to activate PPAR α and γ reporter activity in 3T3-L1 cells and to bind to PPAR γ and δ ligand binding domain. Therefore, in accordance with the present invention, abscisic acid and/or punicic acid, and the plants and extracts thereof containing abscisic acid, punicic acid and/or catalpic acid, are believed to be useful in accordance with the present invention, used in combination with Andrographis or used alone, to treat addiction or to prevent relapse to practice of an addiction.

Red clover extract is sometimes used to treat menopausal disorders as an alternative to therapy with synthetic hormones. Mueller et al (2008) demonstrated that red clover extract, and the active substances genistein and biochanin A, are potent activators of PPARγ. Alleva L. et al. (2009) have also described Biochanin A. as a ligand of PPAR alpha and gamma, the active isoflavone of in Trifolium pretense (red clover) and as having anti-inflammatory properties. Therefor, in accordance with the present invention, red clover extract, and the active substances genistein and Biochanin A contained therein, are believed to be useful in accordance with the present invention, used in combination with Andrographis or used alone, to treat addiction or to prevent relapse to practice of an addiction.

Curcumin has been suggested to act as a PPARγ agonist and has been suggested as an inhibitor of inflammation with potential utility in Alzheimer's Disease. [Wang H M et al., (2010)] Therefor, in accordance with the present invention, curcumin is believed to be useful in accordance with the present invention, used in combination with Andrographis or used alone, to treat addiction or to prevent relapse to practice of an addiction.

Cornus kousa F. Buerger ex Miguel, an oriental medicinal plant, has been traditionally used for the treatment of hyperglycemia. Kim d et al. (2011) demonstrated that Cornus kousa leaf extract (CKE) increased PPARγ ligand-binding activity in 3T3-L1 cells, in a dose-dependent manner. Therefore, in accordance with the present invention, Cornus kousa, active substances contained therein and extracts thereof are believed to be useful in accordance with the present invention, used in combination with Andrographis or used alone, to treat addiction or to prevent relapse to practice of an addiction.

Cistus Salvifolius (Cistaceae) is a medicinal plant found in Greek flora. Kuhn C. et al., (2011) report that the bioguided fractionation of Cistus Salvifolius yields PPARγ stimulating metabolites with differing chemical natures. Therefore, in accordance with the present invention, Cistus Salvifolius, active substances contained therein and extracts thereof are believed to be useful in accordance with the present invention, used in combination with Andrographis or used alone, to treat addiction or to prevent relapse to practice of an addiction.

Kuroda M et al. (2010) performed bioassay-guided fractionation of the EtOH extract of licorice (Glycyrrhiza glabra roots), using a GAL-4-PPAR-gamma chimera assay method. Among the isolated compounds, 5′-formylglabridin (5), (2R,3R)-3,4′,7-trihydroxy-3′-prenylflavane (7), echinatin, (3R)-2′,3′,7-trihydroxy-4′-methoxyisoflavan, kanzonol X, kanzonol W, shinpterocarpin, licoflavanone A, glabrol, shinflavanone, gancaonin L, and glabrone all exhibited significant PPAR-gamma ligand-binding activity. Therefore, in accordance with the present invention, Glycyrrhiza glabra roots, active substances contained therein and extracts thereof are believed to be useful in accordance with the present invention, used in combination with Andrographis or used alone, to treat addiction or to prevent relapse to practice of an addiction.

Rozema, E. et al (2012) evaluated 14 extracts of Chinese herbal medicines (CHM) for PPAR γ and activity and other bioactivity. When testing for PPARγ agonistic activity, a significant effect (P<0.05) was observed for apolar extracts of seven out of the fourteen plants used as CHM (Table 2). Moderate to strong PPARγ activity was observed for apolar extracts from Albizia julibrissin, Arisaema sp., Cnidium monnieri, Pinellia ternata and Tribulus terrestris. Therefore, in accordance with the present invention, Albizia julibrissin, Arisaema sp., Cnidium monnieri, Pinellia ternata and Tribulus terrestris, active substances contained therein and extracts thereof are believed to be useful in accordance with the present invention, used in combination with Andrographis or used alone, to treat addiction or to prevent relapse to practice of an addiction.

In one aspect of the invention, the additional therapeutic agent administered with Andrographis is an opioid antagonist or a mixed opioid antagonist/partial agonist. In a particular embodiment, the opioid antagonist is naltrexone. In another particular embodiment, the mixed opioid partial agonist/antagonist is buprenorphine. An opioid antagonist acts on one or more opioid receptors. At least three types of opioid receptors, mu, kappa, and delta opioid receptors, have been reported, and opioid antagonists are generally classified by their effects on the opioid receptors. Opioid antagonists may antagonize central receptors, peripheral receptors or both. Naloxone and naltrexone are commonly used opioid antagonist drugs that are competitive that bind to the opioid receptors with higher affinity than agonists, but that do not activate the receptors. This effectively blocks the receptor, preventing the body from responding to opiates and endorphins.

Many opioid antagonists are not pure antagonists but also produce some weak opioid partial agonist effects, and can produce analgesic effects when administered in high doses to opioid-naive individuals. Examples of such compounds include nalorphine, and levallorphan. However, the analgesic effects from these drugs are limited and tend to be accompanied by dysphoria, most likely due to action at the kappa opioid receptor. Since they induce opioid withdrawal effects in people who are taking, or have previously used, opioid full agonists, these drugs are considered to be antagonists.

Naloxone is one example of an opioid antagonist that has no partial agonist effects. Instead, it is a weak inverse agonist at mu opioid receptors, and is used for treating opioid overdose.

Specific examples of opioid antagonists that may be used according to the invention include alvimopan, binaltorphimine, buprenorphine, cyclazocine, cyclorphan, cypridime, dinicotinate, beta-funaltrexamine, levallorphan, methylnaltrexone, nalbuphine, nalide, nalmefene, nalmexone, nalorphine, nalorphine dinicotinate, naloxone, naloxonazine, naltrendol, naltrexone, naltrindole, oxilorphan, and pentazocine.

In one aspect of the invention, the additional therapeutic agent administered with Andrographis is topiramate or levetiracetam.

In one aspect of the invention, the additional therapeutic agent administered with Andrographis is an antidepressant. In a particular embodiment, the antidepressant is bupropion or sibutramine. Antidepressants are drugs used to treat depression. The three neurotransmitters believed to be involved in depression are serotonin, dopamine, and norepinephrine. Certain types of antidepressants increase the levels of one or more of these neurotransmitters in the brain by blocking their reabsorption.

Several different classes of antidepressants have been identified, including selective serotonin reuptake inhibitors (SSRIs), tricyclic and tetracyclic serotonin and norepinephrine reuptake inhibitors (SNRIs), norepinephrine reuptake inhibitors (NRIs), norepinephrine and dopamine reuptake inhibitors (NDRIs), azaspirones, monoamine oxidase inhibitors (MAOIs), and atypical antidepressants.

SSRIs include, e.g., cericlamine, citalopram, clomipramine, cyanodothiepin, dapoxetine, duloxetine, escitalopram, femoxetine, fluoxetine, fluvoxamine, ifoxetine, imipramine, indalpine, indeloxazine, litoxetine, lofepramine, mianserine, milnacipran, mirtazapine, nefazadone, nortriptyline, paroxetine, sertraline, sibutramine, tomoxetine, trazodone, venlafaxine, and zimeldine.

Amitriptyline, amoxapine, butriptyline, clomipramine, demexiptiline, desipramine, dibenzepin, dimetacrine, dothiepin, doxepin, imipramine, iprindole, lofepramine, maprotiline, melitracen, metapramine, mianserin, mirtazpine, nortriptyline, propizepine, protriptyline, quinupramine, setiptiline, tianeptine, and trimipramine are all tricyclic and tetracyclic antidepressants.

SNRIs include, e.g., amoxapine, atomoxetine, bicifadine, desipramine, desvenlafaxine, duloxetine, maprotiline, milnacipran, nefazodone, reboxetine, sibutramine, and venlafaxine. Nisoxetine, nortriptyline, reboxetine, talsupram, and tomoxetine are all examples of NRIs. NDRIs include, e.g., bupropion, hydroxybupropion, and tesofensine. Azaspirones include, e.g., buspirone, gepirone, ipsapirone, tandospirone, and tiaspirone. Buspirone is an anxiolytic (partial agonist at 5-HT1 autoreceptors) that may be provided with an antidepressant such as an SSRI. Specific MAOIs include, e.g., amiflamine, brofaromine, clorgyline, alpha-ethyltryptamine, iproclozide, iproniazid, isocarboxazid, mebanazine, moclobemide, nialamide, pargyline, phenelzine, pheniprazine, pirlindole, safrazine, selegiline, toloxatone, and tranlcypromine. Atypical antidepressants include, e.g., amesergide, amineptine, benactyzine, bupropion, clozapine, fezolamine, levoprotiline, lithium, medifoxamine, mianserin, minaprine, olanzapine, oxaflozane, oxitriptan, rolipram, teniloxazine, tofenacin, trazodone, tryptophan, and viloxazine.

In one aspect of the invention, the additional therapeutic agent administered with Andrographis is an antiepileptic. In a particular embodiment, the antiepileptic is levetiracetam. The anticonvulsants, also called antiepileptic drugs (AEDs) are a diverse group of drugs used in prevention of the occurrence of epileptic seizures and bipolar disorders. AEDs suppress the rapid and excessive firing of neurons that begins a seizure and/or prevents the spread of the seizure within the brain and offer protection against possible excitotoxic effects that may result in brain damage. Many anticonvulsants block sodium channels, calcium channels, AMPA receptors, or NMDA receptors.

Antiepileptic agents include, but are not limited to, benzodiazepines, barbituates, valproates, GABA agents, iminostilibenes, hydantoins, NMDA antagonists, sodium channel blockers and succinamides. Benzodiazepines include, e.g., alprazolam, chlordiazepoxide, cholrazepate, clobazam, clonazepam, diazepam, halazapam, lorazepam, oxazepam, and prazepam. Barbiturates used as ant-epileptics include, e.g., amobarbital, mepobarbital, methylphenobarbital, pentobarbital, phenobarbital, and primidone. Valproates used as antiepileptics include, e.g., sodium valporate, valproic acid, valproate semisodium, and valpromide. Antiepileptic GABA agents include, e.g., gabapentin, pregabalin, losigamone, pregabalin, retigabine, rufinamide, and vigabatrin. Carbamazepine and oxcarbazepine are examples of iminostilbenes. Hydantoins include, e.g., fosphenyloin sodium, mephenyloin, and phenyloin sodium. NMDA antagonists such as harkoseramide are used as antiepileptics. Sodium channel blockers such as lamotrigine are also anti-epileptic agents. Succinimides include, e.g., ethosuximide, methsuximide, and phensuximide. Other antiepileptic drugs include acetazolamide, briveracetam, CBD cannabis derivative, clomthiazole edisilate, divalproex sodium, felbamate, isovaleramide, lacosamide, lamotrigine, levetiracetam, methanesulphonamide, talampanel, tiagabine, topiramate, safinamide, seletracetam, soretolide, stiripentol, sultiam, valrocemide, and zonisamide.

In one aspect of the invention, the additional therapeutic agent administered with Andrographis is an antiemetic agent. Antiemetics are drugs effective against vomiting and nausea. Antiemetics are typically used to treat motion sickness and the side effects of opioid analgesics, general anesthetics, and chemotherapy. Classifications of antiemetics include, e.g., 5-hydroxytryptamine 3 (5-HT3) receptor antagonists, histamine receptor antagonists, dopamine receptor antagonists, muscarinic receptor antagonists, acetyl choline receptor antagonists, cannabinoid receptor antagonists, limbic system inhibitors, NK-1 receptor antagonists, corticosteroids, tachykinin antagonists, GABA agonists, cannabinoids, benzodiazepines, anticholinergics, and substance P inhibitors. 5-HT3 receptor antagonists include, e.g., alosetron, azasetron, bemesetron, cilansetron, dolasetron, granisetron, indisetron, itasetron, ondansetron, palonosetron, propisetron, ramosetron, renzapride, tropisetron, and zatosetron. Coritcosteroid antiemetics include dexamethasone and methylprednisolone. Lymbic system inhibitors include alprazolam, lorazepam, and midazolam. Dopamine receptor antagonists include diphenhydramine, dronabinol, haloperidol, metoclopramide, and prochlorperazine. NK-1 receptor antagonists used as an antiemetic include aprepitant and morpholine, and an example of a GABA agonist is propofol. Thiethylperazine is a type of histamine receptor antagonist. Cannabinoid receptor antagonists used as antiemetics include dronabinol, nabilone, rimonabant, tanarabout, and tetrahydrocannabinol, as well as marijuana and extracts of marijuana.

Examples of other antiemetics include acetylleucine, monoethanolamine, alizapride, benzquinamide, bietanautine, bromopride, buclizine, chlorpromazine, clebopride, cyclizine, dimenhydrinate, dipheniodol, domperidone, dranisetron, meclizine, methalltal, metopimazine, oxypendyl, pipamazine, piprinhydrinate, scopolamine, thioproperzaine, and trimethobenzamide.

In one aspect of the invention, the additional therapeutic agent administered with Andrographis is a cannabinoid receptor antagonist. The cannabinoid receptors are a class of the G-protein coupled receptor superfamily. Their ligands are known as cannabinoids. There are currently two known subtypes, CB1 which is expressed mainly in the brain, but also in the lungs, liver, and kidney, and CB2, which is mainly expressed in the immune system and in hematopoietic cells. It is also believed that there are novel cannabinoid receptors that is, non-CB1 and non-CB2, which are expressed in endothelial cells and in the CNS. Cannabinoid receptor antagonists may be selective for either the CB1 or CB2 receptor. The present invention contemplates the use of either or both CB1 and CB2 receptor antagonists.

Addictive agents (e.g., alcohol, opiates, Delta(9)-tetrahydrocannabinol (Delta(9)-THC) and psychostimulants, including nicotine) elicit a variety of chronically relapsing disorders by interacting with endogenous neural pathways in the brain. In particular, they share the common property of activating mesolimbic dopamine brain reward systems, and virtually all abused drugs elevate dopamine levels in the nucleus accumbens. Cannabinoid-1 (CB1) receptors are expressed in this brain reward circuit and modulate the dopamine-releasing effects of Delta(9)-THC and nicotine.

Rimonabant (SR141716), a CB1 receptor antagonist, blocks both the dopamine-releasing and the discriminative and rewarding effects of Delta(9)-THC in animals. Although CB1 receptor blockade is generally ineffective in reducing the self-administration of cocaine in rodents and primates, it reduces the reinstatement of extinguished cocaine-seeking behavior produced by cocaine-associated conditioned stimuli and cocaine priming injections. Similarly, CB1 receptor blockade is effective in reducing nicotine-seeking behavior induced by re-exposure to nicotine-associated stimuli. In human clinical trials, rimonabant was shown to block the subjective effects of Delta(9)-THC in humans and prevents relapse to smoking in ex-smokers.

Other examples of cannabinoid receptor CB1 antagonists include SR141716A (rimonabant), rosanabant, taranabant and CP-945598.

### F. METHODS OF TREATING AND PREVENTING RELAPSE

Relapse use, or reinstatement, refers to the process of returning to the use of alcohol or another addictive agent or the practice of an addictive behavior after a period of abstinence from, or limited or reduced use of, an addictive agent or practice of an addictive behavior. In certain situations, relapse use of an addictive agent refers to the return to use of an addictive agent by a subject who has undergone physical withdrawal from the addictive agent. Typically, the subject will have undergone physical withdrawal from the addictive agent during a period of non-use or limited or reduced use of the addictive agent. In one embodiment, relapse use occurs in a subject who has previously undergone a treatment regime with an effective amount of an antiaddiction agent to reduce or eliminate use of an addictive agent, but who is no longer using an effective amount of the antiaddiction agent. Antiaddictive agents include any and all agents used to treat or prevent addiction or withdrawal symptoms.

Alcoholism, like many other addictions, is a chronic relapsing disorder characterized by high recidivism rates. Two major factors triggering relapse behaviour are stress and environmental conditioning experiences (O'Brien et al. 1997; Monti et al. 1993; Shaham et al. 1995), which probably facilitate relapse to alcohol-seeking via distinct brain mechanisms. For example, activation of the mesolimbic dopamine system via an opioid-dependent mechanism (or via direct alterations in dopamine transmission in the basolateral nucleus of amygdala) seems to mediate the effect of drug-associated cues (Liu and Wiess 2002; Ciccocioppo et al. 2001), and, extrahypothalamic CRF within the bed nucleus of the stria terminalis and median raphe nucleus is likely to mediate stress-induced reinstatement of drug-seeking behaviour (Erb et al 1998; Shaham et al. 1995; Le et al. 2000).

Several lines of evidence suggest that molecular mechanisms underlying relapse to addiction are common to different classes of drugs of abuse. Drug craving and loss of control over drug taking behaviour associated to relapse are under the direct influence of stress and environmental conditioning stimuli; the two major factors affecting resumption to drug use.

Chronic drug abuse produces neuroadaptive changes not only within systems implicated in the acute reinforcing effects of ethanol, but also within other motivational systems, notably brain stress-regulatory mechanisms. Stress has an established role in the initiation and maintenance of drug abuse, and is a major determinant of relapse in abstinent individuals (Brown et al. 1995; Marlatt et al. 1985; McKay et al. 1995; Wallace 1989). The significance of stress in drug-seeking behaviour has also been amply documented in the animal literature. Physical, social, and emotional stress can facilitate acquisition or increase self-administration of cocaine (Goeders et al. 1995; Haney et al. 1995; Ramsey and VanRee 1993; Ahmed and Koob 1997), heroin, (Shaham and Stewart 2004), and ethanol (Nash et al. 1998; Mollenauer et al. 1993; Blanchard et al. 1987; Higley et al. 1991)) in rodents and nonhuman primates. Stressful stimuli have also been shown to elicit reinstatement of cocaine, heroin, and ethanol-seeking behaviour in drug-free animals following extinction (Ahmed and Koob 1997; Shaham 1993; Shaham and Stewart 1995; le et al. 1998) and these findings provide experimental support for a role of stress in relapse.

Traditionally, stress-related drug-seeking behaviour has been thought to be mediated via activation of the hypothalamic-pituitary-adrenal (HPA) axis. However, growing evidence suggests that the non-neuroendocrine corticotropin-releasing factor (CRF) system in the central nucleus of the amygdala (CeA) may play a significant independent role in the regulation of addictive behaviour associated with stress. The CeA is rich in CRF immunoreactive cell bodies, terminals, and receptors, and this neuronal CRF system has been implicated in the mediation of behavioural and emotional responses to stressful stimuli (Dunn and Berridge 1990; Koob et al. 1994). For example, immobilization stress elevates extracellular CRF levels in the CeA (Merlo Pich et al. 1995; Merali et al. 1998) while intra-CeA injection of the CRF receptor antagonist, α-helical CRF9-41, reduces behavioural signs of anxiety produced by social and environmental stressors (Heinrichs et al. 1992; Swiergiel et al. 1993). Anxiety and stress-like symptoms are central to drug and alcohol withdrawal syndromes. Considering the evidence on a role of CRF neurons in the CeA in the regulation of emotional and anxiogenic effects of stress, it is likely that anxiogenic and stress-like consequences of withdrawal from drugs of abuse may be mediated by the CRF system in the CeA as well.

Changes in the regulation of the activity of the CRF system within the CeA may represent a critical neuroadaptive mechanism responsible for the development of dependence and compulsive drug-seeking behaviour.

The data discussed above identify neuroadaptive changes in brain circuitries and perturbations in stress systems as an important element in compulsive drug-seeking behaviour and dependence. Another important factor in the long-lasting addictive potential of drugs of abuse is the conditioning of their rewarding actions with specific environmental stimuli. Environmental cues repeatedly associated with the subjective effects of drugs of abuse including alcohol can evoke drug craving (Childress et al. 1988; Ehrman et al. 1992; Monti et al. 1993; Pomerleau et al. 1983; Stormark et al. 1995) or elicit automatic behavioural responses (Miller and Gold 1994; Tiffany and Carter 1998) that ultimately may lead to relapse. Learned responses to drug-related stimuli may, therefore, contribute critically to the high rates of relapse associated with cocaine and other drug addiction.

Data from operant response-reinstatement models developed to investigate drug-seeking behaviour associated with exposure to drug-related environmental cues in rats indicate that discriminative stimuli predictive of cocaine (Weiss et al. 2000), ethanol (Katner et al. 1999; Katner and Weiss 1999), or heroin (Gracy et al. 2000) availability reliably elicit strong recovery of extinguished drug-seeking behaviour in the absence of further drug availability. The response-reinstating effects of these stimuli show remarkable resistance to extinction with repeated exposure and, in the case of cocaine, can still be observed after several months of forced abstinence. Additionally, in the case of ethanol, drug-seeking behaviour induced by ethanol-predictive discriminative stimuli was found to be enhanced in genetically alcohol-preferring P rats compared to Alcohol Nonpreferring (NP) and nonselected Wistar rats (Weiss and Ciccocioppo 1999). This observation demonstrates that genetic predisposition toward heightened ethanol intake is reflected also by a greater susceptibility to the motivating effects of ethanol cues (i.e., enhanced drug-seeking under conditions where behaviour is not directly reinforced by ethanol itself). Together, these findings strongly support the hypothesis that learned responses to drug-related stimuli are a significant factor in long-lasting vulnerability to relapse.

In humans, relapse risk involves multiple determinants that are likely to interact. For example, exposure to drug cues may augment vulnerability to relapse imparted by protracted withdrawal symptoms resulting from neuroadaptive changes in dependent individuals. Interactive effects exacerbating relapse risk may also exist between the motivating effects of stress and drug-related cues. Recent work addressing these issues has confirmed that additive interactions between the response-reinstating effects of ethanol-associated cues and stress can indeed be demonstrated, and that these effects are enhanced in rats with a history of ethanol dependence (Liu and Weiss 2000).

In experimental laboratories, reinstatement of drug seeking is obtained with administration of the α-2 adrenoreceptor antagonist yohimbine, which, increasing brain noradrenaline cell firing and release, acts as a pharmacological stressor. Footshock stress and yohimbine-induced reinstatement of drug-seeking behaviours both represent valid experimental models to investigate stress-induced alcohol relapse (Lee et al. 2004; Le et al. 2000).

Accordingly, the present invention provides treatment methods and drug combinations that protect individuals from the effects of more than a single environmental risk factor (i.e., stress and environmental conditioning factors).

In one embodiment, the present invention provides a method of treating or preventing stress-induced relapse use of an addictive agent, comprising providing Andrographis to a subject who has undergone physiological withdrawal from an addictive agent.

In a related embodiment, the invention includes a method of treating or preventing relapse use of an addictive agent or practice of an addictive or compulsive behavior, comprising providing an effective amount Andrographis to a subject who previously reduced or eliminated use of an addictive agent or practice of an addictive or compulsive behavior in response to exposure to an effective amount of another antiaddiction treatment, wherein the subject is no longer exposed to an effective amount of the antiaddiction treatment. The antiaddiction treatment may be an antiaddiction drug or may be a non-pharmacologic therapy such as counseling, psychotherapy or hypnosis therapy. The relapse use may be triggered by stress.

In certain embodiments, the subject is no longer exposed to an effective amount of an antiaddiction agent because the subject has become tolerant to the agent, such that the blood plasma concentration of the antiaddiction agent that was previously effective in treating the addiction is no longer effective. In other embodiments, the subject is no longer exposed to an effective amount of an antiaddiction agent because the subject is now exposed to a lower blood plasma concentration of the antiaddiction agent, and this lower blood plasma concentration is not effective.

In certain embodiments of the methods of the present invention, the subject has undergone a period of abstinence from, or limited or reduced use of, the addictive agent or practice of the addictive or compulsive behavior. This period of abstinence or limited or reduced use may be, e.g., at least 24 hours, at least 48 hours, at least 3 days, at least 5 days, at least one week, at least 2 weeks, at least 1 month, at least 2 months, at least 4 months, at least 6 months, at least 9 months, at least one year, at least 2 years, or at least 5 years.

In another embodiment, the present invention includes a method of treating or preventing relapse use of an addictive agent, comprising providing Andrographis and an additional therapeutic agent, as described above, to a subject who has undergone physiological withdrawal from the addictive agent.

While the methods of the present invention may be practiced in subjects addicted to a single addictive agent, they may also be used in subjects addicted to two or more addictive agents. Similarly, while these methods may be used to prevent relapse use of the addictive agent from which the subject has undergone withdrawal, they may also be adapted to prevent relapse use or the commencement of use of an addictive agent different than the one from which the subject has undergone physiological withdrawal.

### G. METHODS OF REDUCING WITHDRAWAL SYMPTOMS AND TREATING DEPRESSION/ANXIETY

Withdrawal, also known as withdrawal/abstinence syndrome, refers to the characteristic signs and symptoms that appear when a drug or addictive agent that causes physical dependence is regularly used for a long time and then suddenly discontinued or decreased in dosage. Withdrawal symptoms can vary significantly among individuals, but there are some commonalities. Brain dysfunction associated with withdrawal is often characterized by depression, anxiety and craving, and, if extreme, can help drive the individual to continue the drug despite significant harm—the definition of addiction—or even to suicide.

Increased heart rate and/or blood pressure, sweating, and tremors are common signs of withdrawal. More serious symptoms such as confusion, seizures, and visual hallucinations indicate a serious emergency and the need for immediate medical care. Alcohol, opiates, benzodiazepines, and barbiturates are the only commonly abused substances that can be fatal in withdrawal. Abrupt withdrawal from other drugs, such as nicotine or psychostimulants, can exaggerate mild to moderate neurotoxic side effects due to hyperthermia and generation of free radicals, but life-threatening complications are very rare.

The present invention includes a method of reducing one or more withdrawal symptoms associated with reduced or discontinued use of an addictive agent, comprising providing an effective amount of Andrographis to a subject undergoing physiological withdrawal from an addictive agent. In particular embodiments, the addictive agent is alcohol, an opioid agonist, such as morphine, or nicotine.

Andrographis may be provided to the subject before the subject begins withdrawal and/or during the withdrawal process. In a related method, a subject is provided with Andrographis over a period of time during which the subject uses a reduced amount of an addictive agent. For example, the subject may begin Andrographis at the same time that they cease using or begin using a reduced amount of an addictive agent. In one embodiment, the subject uses a step-wise reduced amount of an addictive agent, such as nicotine, at the same time as Andrographis, until physical withdrawal is completed. The subject may then discontinue use of Andrographis or continue use of Andrographis to prevent relapse. Therefore, in related embodiments, the present invention contemplates delivering an addictive agent in combination with Andrographis, e.g., to reduce the likelihood of developing addiction, or to reduce withdrawal symptoms. In particular embodiments, Andrographis is delivered in combination with nicotine or an opioid agonist. Andrographis and the addictive agent may be delivered separately or in a single formulation or via a single delivery means. For example, both nicotine and an extract of Andrographis may be delivered via a transdermal patch, an oral lozenge, or a chewing gum delivery system. Transdermal patches, oral lozenges, and chewing gum containing nicotine are frequently used for the delivery of nicotine to subjects attempting to reduce nicotine use. By including Andrographis in combination with the nicotine in the transdermal patch, lozenge, or chewing gum, or by mixing Andrographis with tobacco in a cigarette, cigar, pipe tobacco mix, chewing tobacco mix or snuff mix, it is believed that the subject will suffer less nicotine withdrawal symptoms. In addition, this may facilitate greater compliance and more rapid reduction in nicotine use. The same principal applies to other addictive agents, including, e.g., opioid agonists.

In one particular embodiment, the addictive agent is nicotine, and the subject reduces or discontinues use of nicotine over a period of time during which the subject is provided with Andrographis, alone or in combination with another therapeutic agent. Andrographis and nicotine combinations in the form of a transdermal patch, lozenge, chewing gum or other delivery vehicle may be prescribed in reducing dosages, or decreasing dosages may be kitted together, to permit tapering off of use of the drug product.

### H. PHARMACEUTICAL COMPOSITIONS, ROUTES OF ADMINISTRATION, UNIT DOSAGE FORMS, AND KITS

The present invention provides methods of using Andrographis, alone or in combination with one or more additional therapeutic agents, such as Omega-3 fatty acids, opioid antagonists, antidepressants, antiepileptics, antiemetics, and CB1 receptor antagonists. Thus, the present invention further includes compositions comprising Andrographis in a nutraceutical base, and Andrographis in combination with one or more additional therapeutic agents, such as Omega-3 fatty acids, opioid antagonists, mixed opioid antagonists/partial agonist, antidepressents, antiepileptics, antiemetics, CRF 1 receptor antagonists and CB1 receptor antagonists. In specific embodiments of the present invention, the following compositions for use in the methods of the present invention for the treatment of addictions are provided:

Andrographis paniculata in dried, fresh, powdered or shredded form;

an extract of Andrographis paniculata in solution in a solvent such as water, ethanol or an edible oil;

the dry solute from an extract solution of Andrographis paniculata;

Andrographis paniculata in a nutraceutical base;

an extract of Andrographis paniculata in a nutraceutical base;

one or more of dehydroandrographolide, andrographolide or neoandrographolide, in isolated form or as part of a botanical extract;

one or more of dehydroandrographolide, andrographolide or neoandrographolide in a nutraceutical base;

an extract of one or more of dehydroandrographolide, andrographolide or neoandrographolide in a solvent such as water, ethanol or an edible oil;

any of the above compositions, also including one or more polyunsaturated fatty acids selected from eicosapentaenoic acid, conjugated linoleic acid and/or docosahexaenoic acid, and suitable including the Omega-3 fatty acid combination of eicosapentaenoic acid, conjugated linoleic acid docosahexaenoic acid;

any of the above compositions, further including an additional therapeutic agent such as an opioid antagonist, mixed opioid antagonists/partial agonist, antidepressent, antiepileptic, antiemetic, CRF 1 receptor antagonist or CB1 receptor antagonist; and

any of the above compositions, further including an addictive agent, such as alcohol, an opioid agonist, marijuana, nicotine or a psychostimulant.

As used herein, the term “nutraceutical base” includes one or more of water, saline, phosphate-buffered saline, edible oils, glycerin, ethanol, fruit juice, tea or brewed tea, coffee or brewed coffee, inert solid fillers such as corn starch, starch, sucrose, talc, gelatin, methylcellulose, and magnesium stearate, gums and any other edible liquid, solid, gas or gel.

Such compositions may be in any suitable form. For instance, a composition comprising Andrographis may be formulated for delivery as a tablet, in a capsule, powder or granules, in a gum, in a tincture, in a liquid dosage form, in a transdermal patch, in a lozenge, in a tea cachet, or in a nasal inhalant.

Dosages of Andrographis in the compositions of the present invention may be readily determined depending upon the route of administration, in order to provide a suitable dosage over the course of administration. In one embodiment, Andrographis paniculata is administered daily at a dose of between 1 mg/kg and 1,000 mg/kg, more suitably between 10 and 750 mg/kg, more suitably between 15 and 450 mg/kg. In another embodiment, an active substance of Andrographis paniculata, such as andrographolide, dehydroandrographolide, neoandrographolide or a combination of these active substances may be administered at a daily dose of between 1 mg/kg and 100 mg/kg, more suitably between 5 and 10 mg/kg. Doses of Andrographis may be administered more than once per day, e.g., every four, eight or twelve hours, or may be administered every other day or weekly.

Compositions of the present invention may further include excipients such as preservatives, stabilizers, dyes and flavoring agents. For example, sodium benzoate, sorbic acid and esters of p-hydroxybenzoic acid may be added as preservatives. In addition, antioxidants and suspending agents may be used.

Pharmaceutical compositions of the invention are generally formulated so as to allow the active ingredients contained therein to be bioavailable upon administration of the composition to a subject. Compositions that will be administered to a subject may take the form of one or more dosage units, where for example, a tablet, capsule or cachet may be a single dosage unit, and a container comprising a combination of agents according to the present invention in aerosol form may hold a plurality of dosage units. The compositions may be made for oral administration, for instance as tablets or capsules, but also may be in the form of aqueous suspensions or solutions, suppositories, slow release forms, for example employing an osmotic pump, transdermal patch, or the like.

In particular embodiments, the composition comprising Andrographis and another therapeutic or addictive agent is administered in one or more doses of a tablet formulation, typically for oral administration. The tablet formulation may be, e.g., an immediate release formulation, a controlled release formulation, or an extended release formulation, e.g., a depot formulation. In particular embodiments, extended release formulations of the invention release at least 80% of the active ingredients in vivo over a period of greater than 24 hours, greater than 48 hours, greater than one week, greater than one month, or even greater than 2 or 4 months. Extended release formulations of the invention therefore allow for less frequency of dosing to the mammal in need thereof than other more immediate or controlled release formulations.

The present invention further includes unit dosage forms of pharmaceutical compositions comprising a PPARγ agonist and another therapeutic agent. Each unit dosage form comprises a therapeutically effective amount of a pharmaceutical composition of the present invention, when used in the recommended amount. For example, a unit dosage form may include a therapeutically effective amount in a single tablet, or a unit dosage form may include a therapeutically effective amount in two or more tablets, such that the prescribed amount comprises a therapeutically effective amount.

A number of the additional therapeutic agents described herein as useful in combination with Andrographis are approved for human use at particular dosages. The present invention contemplates using these agents at their approved dosages or at other effective dosages.

In one embodiment, the present invention includes a kit comprising unit dosage forms of Andrographis and unit dosage forms of nicotine. In one embodiment, the unit dosage forms of nicotine comprise a plurality of different unit dosage forms of nicotine, wherein the different dosage forms of nicotine represent decreasing amount that may be taken one after the other over a period of time, so as to overcome addiction and effectuate withdrawal from the nicotine. The unit dosage forms of nicotine may be present, e.g., in the form of a transdermal or skin patch, gum, or a lozenge.

### EXAMPLES

The following methods were used in the below Examples 1-5, which describe a number of studies performed to demonstrate the effect of Andrographis paniculata and andrographolide on addiction and the prevention of relapse.

Animals

Male genetically selected alcohol-preferring rats were used. They were bred at the Department of Experimental Medicine and Public Health of the University of Camerino (Marche, Italy) for 64 generations from Sardinian alcohol-preferring (sP) rats of the 13th generation, provided by the Department of Neurosciences of the University of Cagliari (Colombo et al, 2006). They are referred to as Marchigian sP (msP) rats (Ciccocioppo et al, 2006). At the time of the experiments their body weight ranged between 300 and 350 g. They were kept in single cages in a room with a reverse 12:12 h light/dark cycle (lights off at 9:30 a.m.), temperature of 20-22° C. and humidity of 45-55%. Rats were offered free access to tap water and food pellets (4RF18, Mucedola, Settimo Milanese, Italy), except when noted. All the procedures were conducted in adherence with the European Community Council Directive for Care and Use of Laboratory Animals and the National Institutes of Health Guide for the Care and Use of Laboratory Animals.

Drugs

A. paniculata was a generous gift of Dr. Nicotra (EPO S.r.1, Milano) and was formulated in a vehicle composed of: 1% methylcellulose, 1% Tween 80 and 98% distilled water. It was administered orally (0, 15, 150 and 450 mg/kg) at a volume of 2 mg/kg.

Andrographolide was purchased from Sigma Aldrich (Sigma Chemical Co., St. Louis, Mo., USA), with purity ≧98%. It was dissolved in 5% dimethyl sulphoxide (DMSO) and administered i.p. (5-10 mg/kg). The injection volume was 1 ml/kg.

GW9662, purchased from Tocris (Bristol, UK), was dissolved in 10% DMSO and 3% Tween 80 and the final volume (5 μg/μl) was adjusted adding distilled water. GW9662 was injected ICV in a volume of 1 μl per rat by means of a stainless-steel injector 2.5 mm longer than the guide cannula, so that its tip protruded into the ventricle.

Yohimbine, purchased from Sigma-Aldrich (Chemical Co., St. Louis, Mo., USA), was dissolved in distilled water and injected i.p. (intraperitoneally) in a volume of 1 ml/kg.

Alcohol solution was prepared fresh every day diluting alcohol 95% (v/v) (Sigma-Aldrich, Chemical Co., St. Louis, Mo., USA) in tap water.

Intracranial Surgery

A 22-gauge guide cannula for intracerebroventricular (ICV) drug injections was stereotaxically implanted into the lateral cerebroventricle with the following coordinates: anteroposterior (AP)=1.0; lateral (L)=1.8; ventral (V)=2.0 with reference to bregma.

Two-Bottle Choice Paradigm

A widely used method for testing fluid preference is the two-bottle choice paradigm, in which water and 10% (v/v) alcohol solution were offered in graduated drinking tubes equipped with metallic drinking spouts and their consumption was measured by reading the volume consumed from the graduated tubes. Food intake was measured by weighing the food containers and taking into account spillage. Alcohol, water and food intakes are expressed as g/kg to reduce the influence of differences in body weight.

Operant Training

The study was conducted using standard operant chambers (Med Associate, St Albans, Vt.) located in sound-attenuating, ventilated environmental cubicles. Each chamber was equipped with a drinking reservoir (volume capacity: 0.30 ml) positioned 4 cm above the grid floor in the centre of the front panel of the chamber, and two retractable levers located 3 cm to the right or to the left of the drinking receptacle. Auditory and visual stimuli were presented via a speaker and a light located on the front panel. A microcomputer controlled the delivery of fluids, presentation of auditory and visual stimuli, and recording of the behavioral data.

Statistical Analysis

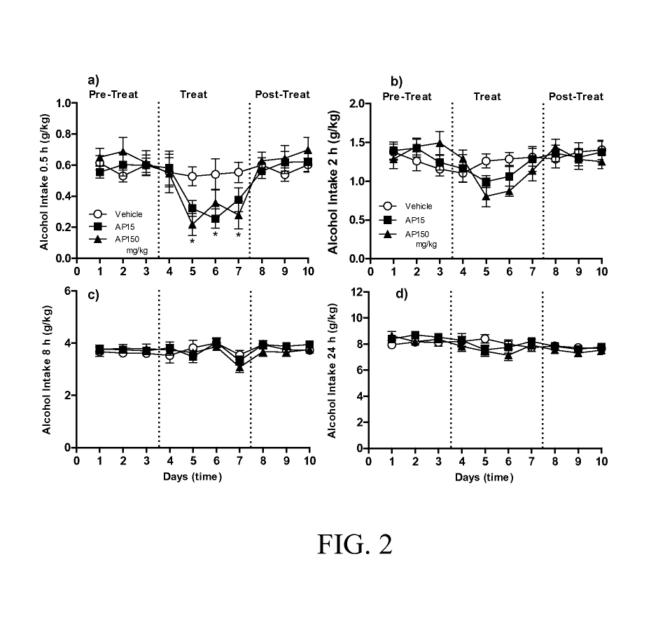

For the study on alcohol drinking and reinstatement analysis of variance (ANOVA) of the results was used followed by Newman Keuls post-hoc tests when appropriate. In particular, the effects of A. paniculata and andrographolide on alcohol drinking was analysed by means of a two-way ANOVA with one factor between (treatment) and one factor within (time). For reinstatement experiments, discrimination was analysed by means of a two-factor ANOVA with one factor within (time) and one factor between (self-administration condition). Differences among responses during the extinction and reinstatement sessions were analysed in the vehicle-treated group by one-way within subjects ANOVA. Reinstatement and drug effects were analysed separately by means of a one factor (reinstatement, or doses) within subject ANOVA. Statistical significance was determined, and is indicated in the FIGS. 2-9, at levels of *p<0.05, **p<0.01 and ***p<0.001.

### Example 1

### Effect of A. Paniculata on Alcohol Intake in msP Rats

To assess the effect of A. paniculata on voluntary alcohol intake, the inventor used the two-bottle choice paradigm in Marchigian Sardinian (msP) rats, a validated animal model of alcohol abuse (Ciccocioppo et al, 2006; Hansson et al, 2006). MsP rats were trained to drink 10% (v/v) alcohol for 24 hours per day (free choice between water and alcohol) until a stable baseline of alcohol intake was reached (5-7 g/kg/day). Before initiation of treatment, rats were trained to drug administration procedures for several days during which they received vehicles. At this point, one group of animals (N=27) was tested for the effect of A. paniculata (0, 15 and 150 mg/kg) on alcohol intake using a between-subject design in which each group of animals (N=9/group) received a different dose of drug. Treatments were continued for 4 consecutive days, and drug or vehicle were administered twice daily at 12 hours and at 1 hour before the beginning of the dark period of the light-dark cycle.

Alcohol, water and food intake were monitored daily at 0.5, 2, 8 and 24 hours. For all the experiments performed, intakes were recorded for several additional days after the end of the drug treatment period. Based on the results obtained, under identical experimental condition, a subsequent study was carried out using an higher dose of A. paniculata, 450 mg/kg. For this purpose a new cohort of msP rats (N=18) was divided into 2 groups. The first group (N=9) received vehicle, while the second group (N=9) received A. paniculata 450 mg/kg, according to the regime administration described above.

Results

A. paniculata administration significantly reduced the voluntary alcohol intake in msP rats (FIG. 2). Analysis of variance (ANOVA) revealed a significant overall effect of treatment only at 0.5 hour [F(2,24)=8.17, p<0.01]. As shown by post-hoc Newman-Keuls tests, the highest drug dose (A. paniculata 150 mg/kg) significantly reduced alcohol consumption the second and fourth day of treatment (*p<0.05). The lower dose injected 15 mg/kg) was effective only the third day of treatment (*p<0.05). Water and Food consumption were not significantly affected by the drug (data not shown). At the end of the treatment rats gradually recovered from the effect of the drug, and alcohol and food intake progressively returned at pre-treatment levels.

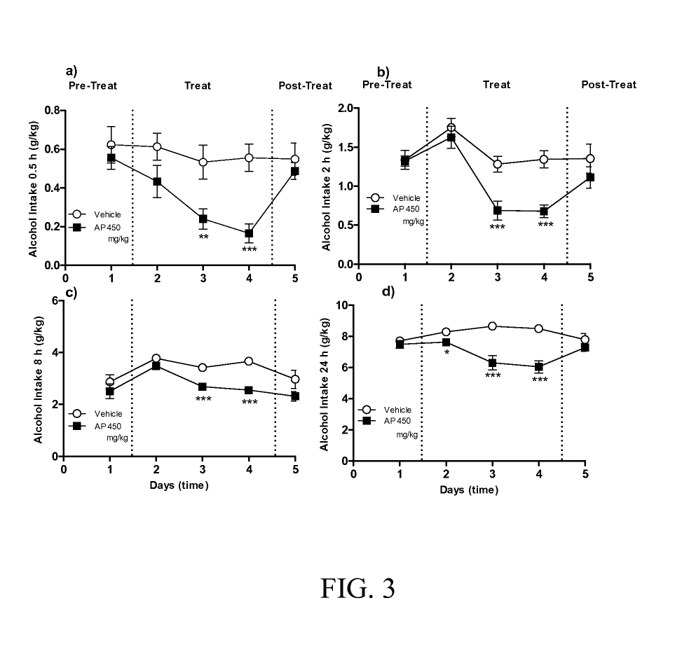

In the second experiment, the overall ANOVA revealed a significant treatment effect of A. paniculata on alcohol intake at 0.5 hours [F(1,16)=14.26; p<0.001], 2 hours [F(1,16)=16.14; p<0.001], 8 hours [F(1,16)=35.87; p<0.001] and 24 hours [F(1,16)=30.34; p<0.001]. Post-hoc Newman-Keuls tests confirmed that this higher dose of drug (450 mg/kg) significantly reduced alcohol consumption starting from the second day of treatment for all the times point tested (FIG. 3). Food consumption was not significantly affected by A. paniculata treatment but a slight trend to an increase was observed (data not shown). Water intake was very low and was not affected by the drug (data not shown). At the end of the treatment rats gradually recovered from the effect of the drug and alcohol and food intake progressively returned at pre-treatment levels (data not shown).

### Example 2

### Effect of A. Paniculata on Yohimbine-Induced Reinstatement of Alcohol Seeking in msP Rats

The experiment consisted of three phases:

Operant Self-Administration Training Phase:

Animals (N=8) were trained to self-administer 10% alcohol (v/v) in 30-minute (30-min) daily sessions on a fixed-ratio 1 schedule of reinforcement, in which each response resulted in delivery of 0.1 ml of fluid. During the first 6 days of training, rats were allowed to lever-press for a 10% (v/v) alcohol solution containing 0.2% (w/v) saccharin solution, on a FR-1 schedule. In the following days, msP rats were trained to self-administer 10% alcohol solution (v/v) on a FR-1, time out 5 second (TO 5 s.) schedule of reinforcement. The TO period was signalled by illumination of a white house light above the right lever for 5 s. 10% (v/v) alcohol self-administration continued until stable baseline of responding was achieved.

Extinction Phase.

After the last alcohol self-administration session, animals were subjected to 30-min extinction sessions. Responses at the lever activated the delivery mechanism but did not result in the delivery of alcohol. The schedule of reinforcement is the same used during alcohol self-administration sessions, except that lever responses were not reinforced. During the last 3 days of extinction, animals were trained to drug administration procedures.

Yohimbine-Induced Reinstatement.

The day after the last extinction session, msP rats were subjected to the 30-min reinstatement test, conducted under the same extinction conditions. To evaluate the effect of A. paniculata on yohimbine-induced reinstatement of Alcohol Seeking behaviour, animals (N=8) were divided into 3 groups (N=2-3/group) and orally treated with A. paniculata (0, 150 and 450 mg/kg) 1 hour before the reinstatement test. Yohimbine (1.25 mg/kg) was given i.p. 30 minute after A. paniculata administration. Animals received all drug treatments according to a counterbalance Latin Square design. A-4 day interval, during which animals were subjected to extinction sessions, was allowed between drug tests (Cippitelli et al, 2008).

Results

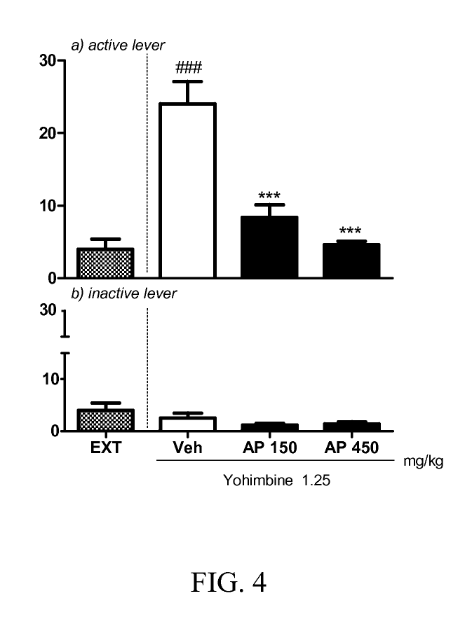

Results showed that a stable baseline of 10% (v/v) alcohol responding was established over nine self-administration days. Following this alcohol self-administration phase, extinction training was initiated and the responses to alcohol progressively decreased. Administration of yohimbine significantly reinstated the operant response for alcohol [F(1,7)=49.34, p<0.001] (FIG. 4). Pre-treatment with A. paniculata significantly reduced the effect of yohimbine [F(2,7)=32.99; p>0.001]. Post hoc analysis demonstrated a significant inhibition of reinstatement following administration of both doses (150 and 450 mg/kg) of A. paniculata tested (p<0.001). Responses at the inactive lever were not influenced by the treatment [F(2,7)=2.14; p>0.05].

### Example 3

### Effect of A. Paniculata on Cue-Induced Reinstatement of Alcohol Seeking in msP Rats

Conditioning Phase.

Using operant chambers (see above), msP rats were trained to discriminate between 10% alcohol and water. The discriminative stimulus for alcohol consisted of the odour of an orange extract (S⁺) whereas water availability (i.e., no reward) was signalled by an anise extract (S⁻). The olfactory stimuli were generated by depositing six to eight drops of the respective extract into the bedding of the operant chamber. In addition, each lever-press resulting in delivery of alcohol was paired with illumination of the chamber's house light for 5 seconds. The corresponding cue during water sessions was a 5 s second white noise tone. Concurrently with the presentation of these stimuli, a 5 second time-out period was in effect, during which responses were recorded but not reinforced. The olfactory stimuli serving as S⁺ or S⁻ for alcohol availability were introduced one minute before extension of the levers and remained present throughout the 30-min sessions. The bedding of the chamber was changed and bedding trays were cleaned between sessions. During the first three days of the conditioning phase the rats were given alcohol sessions only. Subsequently alcohol and water sessions were conducted in random order across training days, with the constraint that all rats received a total of 10 alcohol and 10 water sessions.

Extinction Phase.

After the last conditioning day, rats were subjected to 30-min extinction sessions. During this phase, sessions began by extension of the levers without presentation of the discriminative stimuli. Responses at the lever activated the syringe pumps but did not result in the delivery of liquids or the presentation of the response-contingent cues (house light or tone).

Reinstatement Testing.

Reinstatement tests began the day after the last extinction session. This test lasted 30-min under conditions identical to those during the conditioning phase, except that alcohol and water were not made available. Sessions were initiated by the extension of both levers and presentation of either the alcohol S⁺ or water S⁻ paired stimuli. The respective discriminative stimulus remained present during the entire session and responses at the previously active lever were followed by activation of delivery mechanism and a 5 second presentation of house light in the S⁺ condition or white noise in the S⁻ condition. MsP rats were tested under the S⁻ condition on day 1 and starting from day 2 they were tested under the S⁺ condition. Before initiation of treatment, rats were trained to drug administration procedures for three days during which they received vehicles. To evaluate whether A. paniculata was able to prevent cue-induced reinstatement of alcohol-seeking rats (N=9) were treated per OS with A. paniculata (0, 150, 450 mg/kg) 1 hour before the reinstatement test. Animals received all drug treatments according to a counterbalance Latin square design. A 4-day interval, during which animals remained in their home cages, was allowed between drug tests (Ciccocioppo et al, 2001a, b; Cippitelli et al, 2008; Economidou et al, 2006).

Results

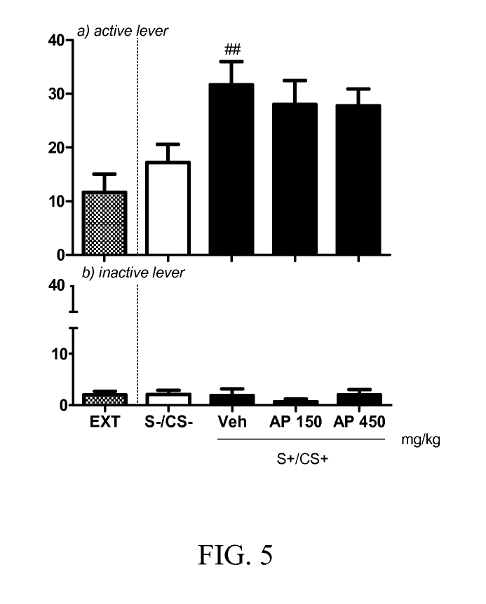

Throughout the conditioning phase, in which animals discriminated between alcohol and water, rats exhibited a strong preference for alcohol. During extinction, lever pressing progressively decreased. In the reinstatement test, the ANOVA showed that cues had a significant overall effect on alcohol seeking [F(2,8)=10.28; p<0.01]. A more detailed analysis showed a robust reinstatement of responding under the S⁺ (<0.01) but not under the S⁻ compared with the last day of extinction. As shown in FIG. 5, conditioned reinstatement of alcohol-seeking was not significantly modified by pre-treatment with A. paniculata [F(2,8)=0.44; p>0.05]. Responses at the inactive lever were not influenced by the treatment [F(2,8)=0.81; p>0.05].

### Example 4

### Effect of Andrographolide on Alcohol Intake in msP Rats

To assess the effect of andrographolide on voluntary alcohol intake, the inventor used the two-bottle choice test in Marchigian Sardinian (msP) rats, a validated animal model of alcohol abuse (Ciccocioppo et al, 2006; Hansson et al, 2006). MsP rats were trained to drink 10% (v/v) alcohol for 24 hours per day (free choice between water and alcohol) until a stable baseline of alcohol intake was reached (5-7 g/kg/day). Before initiation of treatment, rats were trained to drug administration procedures for three days during which they received vehicles. At this point, animals (N=27) were tested for the effect of andrographolide (0, 5 and 10 mg/kg) on alcohol intake using a between-subject design in which each group of animals (N=9/group) received a different dose of drug. Treatments were continued for 3 consecutive days, and drug or vehicle were administered twice daily at 12 hours and at 30 minutes before the beginning of the dark period of the light-dark cycle. Alcohol, water, and food consumption were monitored daily at 0.5, 2, 8 and 24 hours. For all the experiments performed intakes were recorded for three additional days after the end of the drug treatment period.

Results

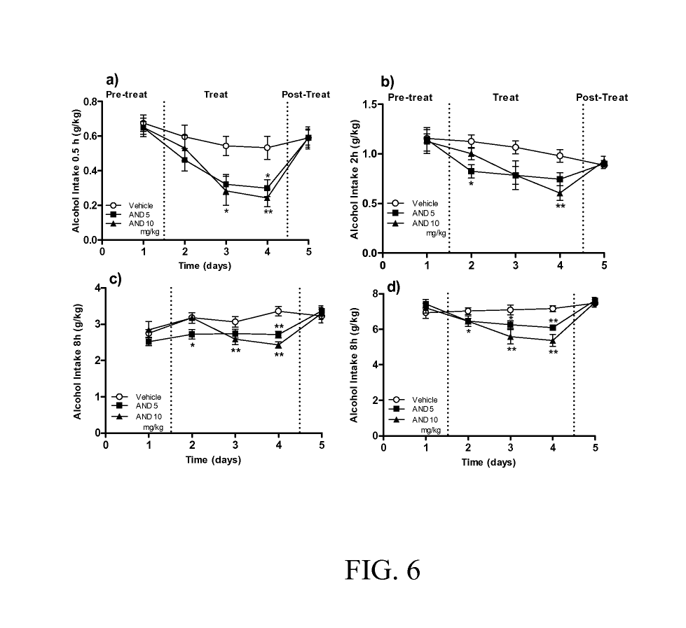

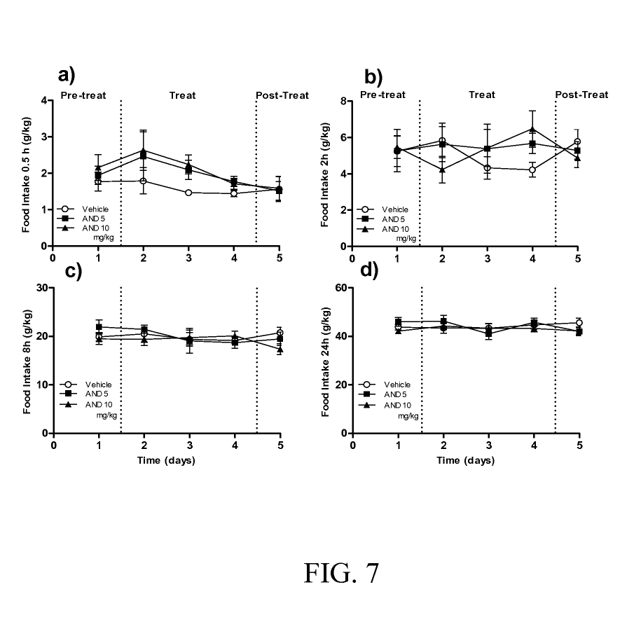

Andrographolide administration significantly reduced voluntary alcohol intake in msP rats (FIG. 6). ANOVA revealed a significant overall effect of treatment [F(2,24)=17.8, p<0.01]. For the highest drug dose (10 mg/kg), the effect appeared from the second day of treatment (Day 3) and remained significant throughout the treatment period (Days 3-4) as shown by Newman-Keuls tests. For the lower dose (5 mg/kg), the effect of treatment increased over time, and a statistical difference from controls was observed the last day of treatment for all the time points. Water and food consumption were not modified by drug treatment, even if a slight trend to an increase was observed at 0.5 hours (FIG. 7) for the higher dose of andrographolide. At the end of treatment, rats gradually recovered from drug effect and alcohol intake progressively returned to pre-treatment levels.

### Example 5

### Effect of Central PPAR Antagonist on Andrographolide-Induced Reduction of Alcohol Intake in msP Rats

To examine whether the effect of andrographolide on alcohol intake is mediated by brain PPAR receptors, another group of msP rats (N=31) was treated ICV with 5 μg/rat of the PPAR antagonist GW9662 and then injected with andrographolide (10 mg/kg) or its vehicle. In a between-subject design each group of animals (N=7-8/group) received a different drug dose for 6 consecutive days. GW9662 or its vehicle were administered twice daily at 12 hours and 30 minutes before the beginning of the dark period of the light-dark cycle. Andrographolide was given immediately after GW9662 administrations.

Alcohol, water, and food intake was recorded at 0.5, 2, 8 and 24 hours from the moment that alcohol was made available.

Results

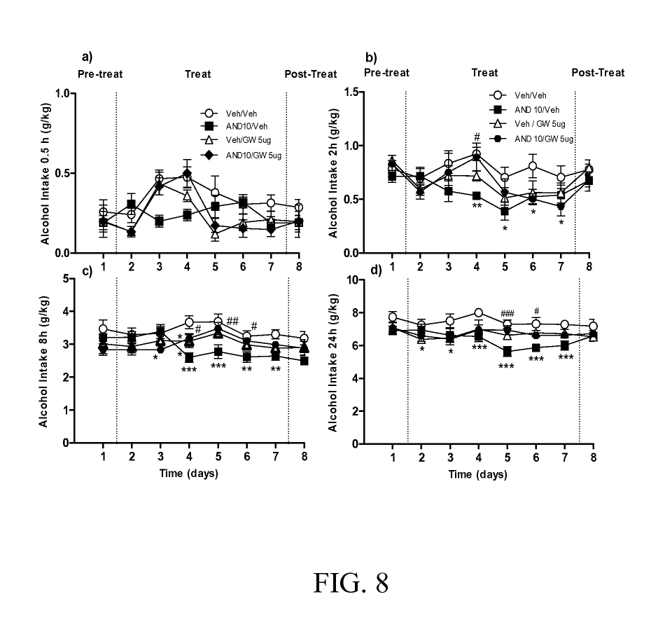

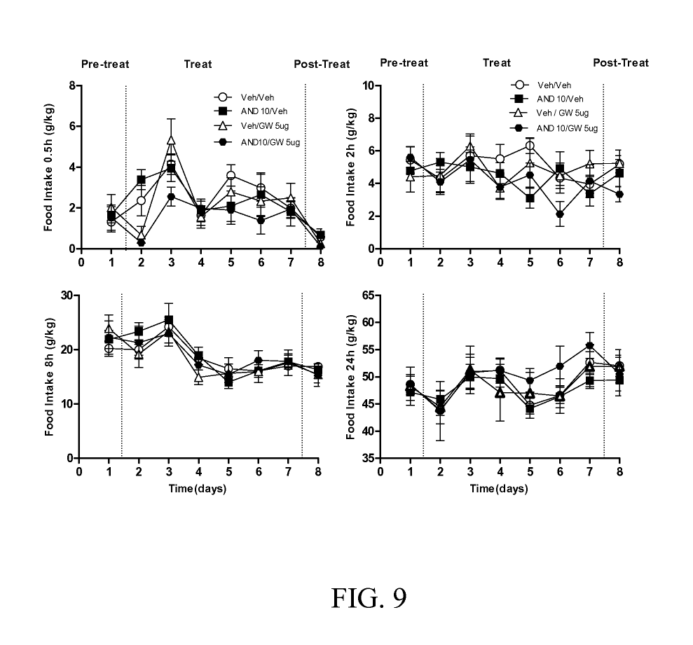

Andrographolide treatment was effective (10 mg/kg) to reduce alcohol drinking at 2 [F(3,27)=3.45, p<0.05], 8 hours [F(3,27)=4.23, p<0.05] and 24 hours [F(3,27)=5.4, p<0.01] (FIG. 8). Newman-Keuls post hoc tests showed that andrographolide treatment reduced alcohol consumption starting from the second day and for the entire duration of treatment and that the effect increased in a time dependent manner. There was no similar effect of the PPAR antagonist GW9662 on alcohol drinking (p>0.05). However, GW9662 blocked the effect of andrographolide at 8 and 24 hours starting from the third day of treatment and until the end of drug administration (FIG. 9). Water intake was not affected by drug treatments (data not shown). ANOVA also revealed no significant overall changes in food intake (data not shown).

### Example 6

### Effect of Andrographis Paniculata on Yohimbine-Induced Reinstatement of Nicotine Seeking

The following study was performed to evaluate the effect of Andrographis Paniculata on yohimbine-induced reinstatement of nicotine seeking

Intravenous Catheterization.

Animals are anesthetized by intramuscular injection of 100-150 μl of a solution containing tiletamine cloridrate (58.17 mg/ml) and zolazepam cloridrate (57.5 mg/ml). For IV surgery, incisions are made to expose the right jugular vein. A catheter made from micro-renathane tubing (I.D.=0.020 inches, O.D.=0.037 inches) is subcutaneously positioned between the vein and the back. After insertion into the vein, the proximal end of the catheter is anchored to the muscles underlying the vein with surgical silk. The distal end of the catheter is attached to a stainless-steel cannula bent at a 90° angle. The cannula is inserted in a support made by dental cement on the back of the animals covered with a plastic cap. For one week after surgery, rats are daily treated with 0.2 ml of the antibiotic Sodium Cefotaxime (262 mg/ml). For the duration of the experiments catheters are daily flushed with 0.2-0.3 ml of heparinized saline solution. Body weights are monitored every day and catheter patency is confirmed approximately every 3 days with an injection of 0.2-0.3 ml of thiopental sodium (250 mg/ml) solution. Patency of the catheter is assumed if there is an immediate loss of reflexes.

Self administration experiments begin 1 week after the post surgery recovery.

Drug Injections.

Nicotine (Sigma Aldrich) is dissolved in saline (0.03 mg/0.1 ml) and given intravenously (IV). Yohimbine Hcl was dissolved in distilled water and given intraperitoneally (IP) at dose of 1.25 mg/kg/ml 60 minutes after Andrographis injection (30 minutes before the relapse test)

Self Administration Apparatus.

The self-administration stations consist of operant conditioning chambers (Med Associate Inc) enclosed in sound-attenuating, ventilated environmental cubicles. Each chamber is equipped with two retractable levers located in the front panel of the chamber. Nicotine is delivered by a plastic tube that was connected with the catheter before the beginning of the session. An infusion pump is activated by responses on the active lever, while responses on the inactive lever are recorded but do not result in any programmed consequences. Activation of the pump results in the delivery of 0.1 ml of fluid (0.03 mg nicotine dose). An IBM compatible computer controls the delivery of fluids and recording of the behavioral data.

Yohimbine Stress Induced Reinstatement of Nicotine Seeking.

Reinstatement of drug seeking is obtained with administration of the α-2 adrenoceptor antagonist yohimbine, a compound known to increase the firing of noradrenergic neurons (Aghajanian et al., 1982) and the release of norepinephrine (Abercrombie et al., 1988). It therefore acts as a pharmacological stressor (Charney et al 1983; Holmberg et al 1962, Lee et al 2004; Le et al., 2005), and will be used to investigate the effect of Andropraphis Paniculata on stress-induced nicotine seeking

Training Phase.

Wistar rats are trained to self-administration of nicotine in 2-hour daily sessions under an FR1 schedule of reinforcement, until a stable baseline of responding is reached. During the infusion, a stimulus house light is turned on for 20 s (time out; TO). Lever presses during the TO period are counted, but do not lead to further infusions.

Extinction Phase.

After the last nicotine self-administration session, animals are subjected to once daily 60-min extinction sessions until lever responding decrease to an extinction level (10±5 right lever presses). During extinction responses, the active lever activates the delivery mechanism but does not result in the delivery of nicotine.

Effect of Andrographis on Yohimbine Stress-Induced Reinstatement.

The day after the last extinction session, in a within subject counterbalanced design animals are given Andrographis Paniculata extract OS at the doses of 0, 150; 450 mg/kg, given 1 hour prior to administration of yohimbine (1.25 mg/kg) or its vehicle. 30 minutes following the administration of yohimbine, animals (N=15) are placed in the self-administration apparatus, and lever pressesis recorded for 2 hours. In a counterbalance Latin square design, each animal receives all drug treatments. A 2-day interval, during which animals are subjected to extinction sessions, occurs between each drug test.

Results

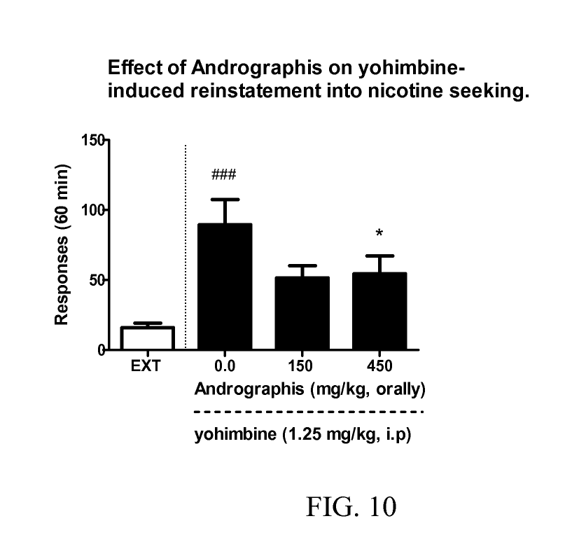

Due to violation of normality distribution of data statistical analysis is conducted using a nonparametric test by comparing multiple dependent samples (Friedman ANOVA & Kendall's concordance). Comparisons are conducted by Wilcoxon matched pairs test showing significance between veh and 450 mg/kg group (see FIG. 10). One way ANOVA revealed a significant reinstatement of responding in yohimbine treated rats compared to extinction (p<0.001).

These results demonstrate the efficacy of A. paniculata as a natural PPARγ associated ligand in decreasing alcohol intake as well as relapse-like behavior, a result that, despite tolerability and safety profile of synthetic PPARγ ligands, may offer important advantages if compared to the treatment with synthetic PPARγ agonists. The reduced alcohol consumption is also observed by using the major and the most active ingredient of A. paniculata, andrographolide, to ensure the reliability of this effect. Finally, it is worth noting that the decreased alcohol consumption induced by andrographolide is blocked by the selective antagonism of centrally expressed PPARγ, indicating that this effect is PPARγ-mediated.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

Abercrombie E D, Keller R W, Jr., Zigmond M J (1988) Characterization of hippocampal norepinephrine release as measured by microdialysis perfusion: pharmacological and behavioral studies. Neuroscience 27: 897-904
Abu-Ghefreh A A, Canatan H, Ezeamuzie C I (2009) In vitro and in vivo anti-inflammatory effects of andrographolide. Int Immunopharmacol 9: 313-8
Adams, A. D., et al. (2003). Amphipathic 3-phenyl-7-propylbenzisoxazoles; human PPAR gamma, delta, and alpha agonists. Bioorg. Med. Chem. Lett. 13(5):931-935.
Adler M W, Geller E B, Chen X, Rogers T J (2005) Viewing chemokines as a third major system of communication in the brain. AAPS J 7: E865-70
Administration, S. A. a. M. H. S. (2003). Substance Abuse and Mental Health Services Administration, Overview of Findings from the 2002 National Survey on Drug Use and Health, Substance Abuse and Mental Health Services Administration Rockville, Md. Office of Applied Studies.
Aghajanian G K, VanderMaelen C P (1982) alpha 2-adrenoceptor-mediated hyperpolarization of locus coeruleus neurons: intracellular studies in vivo. Science 215: 1394-6
Ahmed, S. H. and Koob, G. F., Cocaine- but not food-seeking behavior is reinstated by stress after extinction, Psychopharmacology, 132, 289, 1997.
Airavaara M, Tuomainen H, Piepponen T P, Saarma M, Ahtee L (2007) Effects of repeated morphine on locomotion, place preference and dopamine in heterozygous glial cell line-derived neurotrophic factor knockout mice. Genes Brain Behav 6: 287-98
Alfonso-Loeches S, Pascual-Lucas M, Blanco A M, Sanchez-Vera I, Guerri C (2010) Pivotal role of TLR4 receptors in alcohol-induced neuroinflammation and brain damage. J Neurosci 30: 8285-95
Lleva L. M., et al. (2010) Using Complementary and Alternative Medicines to Target the Host Response During Severe Influenza, eCAM 2010; 7(4) 501510.
Amroyan E, Gabrielian E, Panossian A, Wikman G, Wagner H (1999) Inhibitory effect of andrographolide from Andrographis paniculata on PAF-induced platelet aggregation. Phytomedicine 6: 27-31
Anisman H, Merali Z, Hayley S (2003) Sensitization associated with stressors and cytokine treatments. Brain Behav Immun 17: 86-93
Association, A. P. (1994). Diagnostic and Statistical Manual of Mental Disorders, Fourth Edition. Washington, D.C., American Psychiatric Association.
Aston-Jones G, Kalivas P W (2008) Brain norepinephrine rediscovered in addiction research. Biol Psychiatry 63: 1005-6
Bajetto A, Bonavia R, Barbero S, Schettini G (2002) Characterization of chemokines and their receptors in the central nervous system: physiopathological implications. J Neurochem 82: 1311-29
Bao Z, Guan S, Cheng C, Wu S, Wong S H, Kemeny D M, Leung B P, Wong W S (2009) A novel antiinflammatory role for andrographolide in asthma via inhibition of the nuclear factor-kappaB pathway. Am J Respir Crit Care Med 179: 657-65
Bassaganya-Riera, J. et al. (2011) Treatment of Obesity-Related Complications with Novel Classes of Naturally Occurring PPAR agonists, Journal of Obesity Volume 2011, Article ID 897894, 7 pages.
Bauer S, Patterson P H (2006) Leukemia inhibitory factor promotes neural stem cell self-renewal in the adult brain. J Neurosci 26: 12089-99
Berger J, Moller D E (2002) The mechanisms of action of PPARs. Annu Rev Med 53: 409-35
Blanchard, R. J., Hori, K., Tom, P., and Blanchard, C., Social structure and ethanol consumption in laboratory rats, Pharmacol. Biochem. Behav., 28, 437, 1987.
Breese G R, Knapp D J, Overstreet D H, Navarro M, Wills T A, Angel R A (2008) Repeated lipopolysaccharide (LPS) or cytokine treatments sensitize ethanol withdrawal-induced anxiety-like behavior. Neuropsychopharmacology 33: 867-76
Breese G R, Overstreet D H, Knapp D J, Navarro M (2005b) Prior multiple ethanol withdrawals enhance stress-induced anxiety-like behavior inhibition by CRF1- and benzodiazepine-receptor antagonists and a 5-HT1a-receptor agonist. Neuropsychopharmacology 30: 1662-9
Brower K J (2003) Insomnia, alcoholism and relapse. Sleep Med Rev 7: 523-39
Brown S. A., Vik P. W., Patterson T. L., et al. Stress, vulnerability and adult alcohol relapse. J Studies Alcohol 1995; 56:538.
Chan S J, Wong W S, Wong P T, Bian J S (2010) Neuroprotective effects of andrographolide in a rat model of permanent cerebral ischaemia. Br J Pharmacol 161: 668-79
Charney D S, Heninger G R, Redmond D E, Jr. (1983) Yohimbine induced anxiety and increased noradrenergic function in humans: effects of diazepam and clonidine. Life Sci 33: 19-29
Childress, A. R., Ehrman, R. N., McLellan, A. T., and O'Brien, C. P., Conditioned craving and arousal in cocaine addiction: A preliminary report, in NIDA Research Monograph 81, US Government Printing Office, Washington, D.C., 1988, 74.
Ciccocioppo R, Angeletti S, Weiss F (2001a) Long-lasting resistance to extinction of response reinstatement induced by ethanol-related stimuli: role of genetic ethanol preference. Alcohol Clin Exp Res 25: 1414-9
Ciccocioppo R, Economidou D, Cippitelli A, Cucculelli M, Ubaldi M, Soverchia L, Lourdusamy A, Massi M (2006) Genetically selected Marchigian Sardinian alcohol-preferring (msP) rats: an animal model to study the neurobiology of alcoholism. Addict Biol 11: 339-55
Ciccocioppo R, Martin-Fardon R, Weiss F (2002) Effect of selective blockade of mu(1) or delta opioid receptors on reinstatement of alcohol-seeking behavior by drug-associated stimuli in rats. Neuropsychopharmacology 27: 391-9
Ciccocioppo R., Angeletti S., Sanna P. P., Weiss F., Massi M. Effect of nociceptin/orphanin FQ on the rewarding properties of morphine. Eur J. Pharmacol. 2000; 404: 153-9.
Ciccocioppo R., Katner S, N., Weiss F. Relapse induced by alcohol-associated enviromental stimuli after extinction in rats. Alcohol Clin Exp Res 1999-c; 23(Suppl):52A.
Ciccocioppo R., Panocka I., Polidori C., Regoli D., Massi M. Effect of nociceptin on alcohol intake in alcohol-preferring rats. Psychopharmacology 1999-a; 141:220-4.
Ciccocioppo R., Sanna P. P., Weiss F. Cocaine-predictive stimulus induces drug-seeking behaviour and neural activation in limbic brain regions after multiple months of abstinence: reversal by D(1) antagonists. Proc Natl Acad Sci USA 2001-b; 98(4):1976-81.
Cippitelli A, Cannella N, Braconi S, Duranti A, Tontini A, Bilbao A, Defonseca F R, Piomelli D, Ciccocioppo R (2008) Increase of brain endocannabinoid anandamide levels by FAAH inhibition and alcohol abuse behaviours in the rat. Psychopharmacology (Berl) 198: 449-60
Colombo G, Lobina C, Carai M A, Gessa G L (2006) Phenotypic characterization of genetically selected Sardinian alcohol-preferring (sP) and -non-preferring (sNP) rats. Addict Biol 11: 324-38
Compton, W. M. and N. D. Volkow (2006). Abuse of prescription drugs and the risk of addiction. Drug Alcohol Depend 83 Suppl 1: S4-7.
Compton, W. M. and N. D. Volkow (2006). Major increases in opioid analgesic abuse in the United States: concerns and strategies. Drug Alcohol Depend 81(2): 103-7.
Coon J T, Ernst E (2004) Andrographis paniculata in the treatment of upper respiratory tract infections: a systematic review of safety and efficacy. Planta Med 70: 293-8
Delva, J., J. M. Wallace, Jr., et al. (2005). The epidemiology of alcohol, marijuana, and cocaine use among Mexican American, Puerto Rican, Cuban American, and other Latin American eighth-grade students in the United States: 1991-2002. Am J Public Health 95(4): 696-702.
Deng W L (1985) [Pharmacological studies on thirteen kinds of injections from Andrographis paniculata. I. Antipyretic, anti-inflammatory effects and toxicity]. Zhong Yao Tong Bao 10: 38-42
Dolganiuc A, Bakis G, Kodys K, Mandrekar P, Szabo G (2006) Acute ethanol treatment modulates Toll-like receptor-4 association with lipid rafts. Alcohol Clin Exp Res 30: 76-85
Dunn A J, Swiergiel A H, de Beaurepaire R (2005) Cytokines as mediators of depression: what can we learn from animal studies? Neurosci Biobehav Rev 29: 891-909
Dunn, A. J. and Berridge, C. W., Physiological and behavioural responses to corticotropin-releasing factor administration: is CRF a mediator of anxiety or stress responses?, Brain Res Brain Res Rev, 15, 71, 1990.
Economidou D, Mattioli L, Cifani C, Perfumi M, Massi M, Cuomo V, Trabace L, Ciccocioppo R (2006) Effect of the cannabinoid CB1 receptor antagonist SR-141716A on ethanol self-administration and ethanol-seeking behaviour in rats. Psychopharmacology (Berl) 183: 394-403
Ehrman, R. N., Robbins, S. J., Childress, A. R., and O'Brien, C. P., Conditioned responses to cocaine-related stimuli in cocaine abuse patients, Psychopharmacology, 107, 523, 1992.
Erb Suzanne, Shaham Yavin, Stewart Jane. The Role of Corticotropin-Releasing Factor and Corticosterone in Stress- and Cocaine-Induced Relapse to Cocaine Seeking in Rats. J Neurosci 1998; 18(14):5529-3
Goeders, N. E. and Guerin, G. F., Non-contingent electric shock facilitates the acquisition of intravenous cocaine self-administration in rats, Psychopharmacology, 114, 63, 1994.
Gracy, K. N., Dankiewicz, L. A., Weiss, F., and Koob, G. F., Heroin-specific cues reinstate heroin-seeking behaviour in rats after prolonged extinction, Pharmacol. Biochem. Behav., 65, 489, 2000.
Haney, M., Maccari, S., Le Moal, M., Simon, H., and Piazza, P. V., Social stress increases the acquisition of cocaine self-administration in male and female rats, Brain Res., 698, 46, 1995.
Hanke Orozco J L, Valdivia C L, Burgos Aguilera R A (2006) Composition of Labdane diterpenes extracted from Andrographis paniculata, useful for the treatment of autoimmune diseases, and Alzeheimer disease by activation of PPAR-gamma receptors, United States patent application publication
Hansson A C, Cippitelli A, Sommer W H, Fedeli A, Bjork K, Soverchia L, Terasmaa A, Massi M, Heilig M, Ciccocioppo, R (2006) Variation at the Rat Crhr 1 Locus and Sensitivity to Relapse Into Alcohol Seeking Induced by Environment Stress, Proc Nat Acad Sci USA 1034(41):15236-41.
Hasin D S, Stinson F S, Ogburn E, Grant B F (2007) Prevalence, correlates, disability, and comorbidity of DSM-IV alcohol abuse and dependence in the United States: results from the National Epidemiologic Survey on Alcohol and Related Conditions. Arch Gen Psychiatry 64: 830-42
Hayley S, Poulter M O, Merali Z, Anisman H (2005) The pathogenesis of clinical depression: stressor- and cytokine-induced alterations of neuroplasticity. Neuroscience 135: 659-78
He D Y, McGough N N, Ravindranathan A, Jeanblanc J, Logrip M L, Phamluong K, Janak P H, Ron D (2005) Glial cell line-derived neurotrophic factor mediates the desirable actions of the anti-addiction drug ibogaine against alcohol consumption. J Neurosci 25: 619-28
He J, Crews F T (2008) Increased MCP-1 and microglia in various regions of the human alcoholic brain. Exp Neurol 210: 349-58
Heilig M, Goldman D, Berrettini W, O'Brien C P (2011) Pharmacogenetic approaches to the treatment of alcohol addiction. Nat Rev Neurosci 12: 670-84
Heinrichs, S. C., Merlo Pich, E., Miczek, K. A., Britton, K. T., and Koob, G. F., Corticotropin-releasing factor reduces emotionality in socially defeated rats via direct neurotropic action, Brain Res., 581, 190, 1992.
Higley, J. D., Hasert, M. F., Suomi, S. J., and Linnoila, M., Nonhuman primate model of alcohol abuse: effect of early experience, personality, and stress on alcohol consumption., Proc. Natl. Acad. Sci. USA, 88, 7261, 1991.
Holmberg G, Gershon S, Beck L H (1962) Yohimbine as an autonomic test drug. Nature 193: 1313-4
Holmberg, G., S. Gershon, et al. (1962). Yohimbine as an autonomic test drug. Nature 193: 1313-4.

Iruretagoyena M I, Sepulveda S E, Lezana J P, Hermoso M, Bronfman M, Gutierrez M A, Jacobelli S H, Kalergis A M (2006) Inhibition of nuclear factor-kappa B enhances the capacity of immature dendritic cells to induce antigen-specific tolerance in experimental autoimmune encephalomyelitis. J Pharmacol Exp Ther 318: 59-67
Irwin M R, Olmstead R, Valladares E M, Breen E C, Ehlers C L (2009) Tumor necrosis factor antagonism normalizes rapid eye movement sleep in alcohol
Katner S. N. and Weiss F. Ethanol-associated olfactory stimuli reinstate ethanol-seeking behaviour after extinction and modify extracellular dopamine levels in the nucleus accumbens. Alcohol Clin Exp Res 1999; 23:1751.
Katner, S, N., Magalong, J. G., and Weiss, F., Reinstatement of alcohol-seeking behaviour by drug-associated discriminative stimuli after prolonged extinction in the rat, Neuropsychopharmacology, 20, 471, 1999.
Katsukawa et al. (2011) Citronellol and Geraniol, VComponenets of Rose Oil, Activate Peroxisome Proliferator-Activated Receptor α and γ and Suppress Cyclooxygenase-2 Expression, Biosci. Biotechnol. Biochem. 17 (5) 1010-12.
Kawasaki Y, Zhang L, Cheng J K, Ji R R (2008) Cytokine mechanisms of central sensitization: distinct and overlapping role of interleukin-1beta, interleukin-6, and tumor necrosis factor-alpha in regulating synaptic and neuronal activity in the superficial spinal cord. J Neurosci 28: 5189-94
Kim D, et al. (2011) Cornus kousa F. Buerger ex Miguel Increases Glucose Uptake Through Activation of Peroxisome Proliferator-Activated Receptor γ and Insuin Sensitization, J. Ethnopharmacol 133(2): 803-9 Epub 2010 Nov. 9.
Knapp D J, Whitman B A, Wills T A, Angel R A, Overstreet D H, Criswell H E, Ming Z, Breese G R (2011) Cytokine involvement in stress may depend on corticotrophin releasing factor to sensitize ethanol withdrawal anxiety. Brain Behav Immun 25 Suppl 1: S146-54
Koob G. F., Heinrichs S. C., Menzaghi F., et al. Corticotropin releasing factor, stress and behaviour. Semin Neurosci 1994; 6:221.
Koob G F (2008) A role for brain stress systems in addiction. Neuron 59: 11-34
Koob G F (2010) The role of CRF and CRF-related peptides in the dark side of addiction. Brain Res 1314: 3-14
Koob G F, Le Moal M (1997) Drug abuse: hedonic homeostatic dysregulation. Science 278: 52-8
Koob G F, Le Moal M (2008) Addiction and the brain antireward system Annu Rev Psychol 59: 29-53
Koteswara Rao Y, Vimalamma G, Rao C V, Tzeng Y M (2004) Flavonoids and andrographolides from Andrographis paniculata. Phytochemistry 65: 2317-21
Kreutzberg G W (1996) Microglia: a sensor for pathological events in the CNS. Trends Neurosci 19: 312-8
Kuhn C et al. (2011) Constituents from Cistus Salvifolius (Cistaceae) Activate Peroxisome Proliferator-Activated Receptor-γ but not -α and Stimulate Glucose Uptake by Adipocytes, Planta Med. 77(4):364-53 Epub 2010 Oct. 4.
Kuroda m. et al. (2010) Phenolics for Glycyrrhiza Glabra roots and their PPAR-gamma Ligand-Binding Activity, Bioorg Med Chem 18(2):962-70. Epub 2009 Nov. 26.
Ladenheim B, Krasnova I N, Deng X, Oyler J M, Polettini A, Moran T H, Huestis M A, Cadet J L (2000) Methamphetamine-induced neurotoxicity is attenuated in transgenic mice with a null mutation for interleukin-6 Mol Pharmacol 58: 1247-56
Le A. D., Harding S., Juzytsch W., Watchus J., Shalev U., Shaham Y. The role of corticotrophin-releasing factor in stress-induced relapse to alcohol-seeking behaviour in rats. Psychopharmacology (Berl) 2000; 150(3):317-24.
Le A D, Harding S, Juzytsch W, Funk D, Shaham Y (2005) Role of alpha-2 adrenoceptors in stress-induced reinstatement of alcohol seeking and alcohol self-administration in rats. Psychopharmacology (Berl) 179: 366-73
Lee B, Tiefenbacher S, Platt D M, Spealman R D (2004) Pharmacological blockade of alpha2-adrenoceptors induces reinstatement of cocaine-seeking behavior in squirrel monkeys. Neuropsychopharmacology 29: 686-93
Lee B., Tiefenbacher S., Platt D. M., Spealman R. D. Pharmacological blockade of alpha2-adrenoceptors induces reinstatement of cocaine-seeking behavior in squirrel monkeys. Neuropsychopharmacology. 2004; 29:686-93.
Lin S L, Tsai R Y, Tai Y H, Cherng C H, Wu C T, Yeh C C, Wong C S Ultra-low dose naloxone upregulates interleukin-10 expression and suppresses neuroinflammation in morphine-tolerant rat spinal cords. Behav Brain Res 207: 30-6
Liu X., Weiss F. Reversal of ethanol-seeking behaviour by D1 and D2 antagonists in an animal model of relapse: differences in antagonist potency in previously ethanol-dependent versus nondependent rats. J Pharmacol Exp Ther 2002; 300(3):882-9.
Liu, X. and Weiss, F., Reinstatement of ethanol-seeking behaviour by stress- and drug-related cues in rats with a history of ethanol dependence, Soc. Neurosci. Abstr. 26, 786, 2000.
Lu W (1995) Prospect for study on treatment of AIDS with traditional Chinese medicine. J Tradit Chin Med 15: 3-9
Ludwig A M, Wikler A (1974) “Craving” and relapse to drink. Q J Stud Alcohol 35: 108-30
Ludwig A M, Wikler A, Stark L H (1974) The first drink: psychobiological aspects of craving. Arch Gen Psychiatry 30: 539-47
Marinelli P W, Funk D, Juzytsch W, Harding S, Rice K C, Shaham Y, Le A D (2007) The CRF1 receptor antagonist antalarmin attenuates yohimbine-induced increases in operant alcohol self-administration and reinstatement of alcohol seeking in rats. Psychopharmacology (Berl) 195: 345-55
Marlatt, G. A., Relapse prevention: introduction and overview of the model, in Relapse Prevention: Maintenance Strategies in the Treatment of Addictive Behaviours, Guilford, London, 1985, 3.
McCusker C G, Brown K (1990) Alcohol-predictive cues enhance tolerance to and precipitate “craving” for alcohol in social drinkers. J Stud Alcohol 51: 494-9
McKay, J. R., Rutherford, M. J., Alternan, A. I., Cacciola, J. S., and Kaplan, M. R., An examination of the cocaine relapse process, Drug Alcohol Dep., 38, 35, 1995.
McLellan A T, Lewis D C, O'Brien C P, Kleber H D (2000) Drug dependence, a chronic medical illness: implications for treatment, insurance, and outcomes evaluation. JAMA 284: 1689-95
Merali, Z., McIntosh, J., Kent, P., Michaud, D., and Anisman, H., Aversive and Appetitive Events Evoke the Release of Corticotropin-Releasing Hormone and Bombesin-Like Peptides at the Central Nucleus of the Amygdala, J. Neurosci., 18, 4758, 1998.
Merlo Pich, E., Lorang, M. T., Yeganeh, M., De Fonseca, F. R., Raber, J., Koob, G. F., and Weiss, F., Increase of extracellular corticotropin-releasing factor-like immunoreactivity levels in the amygdala of awake rats during restraint stress and ethanol withdrawal as measured by microdialysis, J. Neurosci., 15, 5439, 1995.
Messer C J, Eisch A J, Carlezon W A, Jr., Whisler K, Shen L, Wolf D H, Westphal H, Collins F, Russell D S, Nestler E J (2000) Role for GDNF in biochemical and behavioral adaptations to drugs of abuse. Neuron 26: 247-57
Mika J (2008) Modulation of microglia can attenuate neuropathic pain symptoms and enhance morphine effectiveness. Pharmacol Rep 60: 297-307
Miller, N. S, and Gold, M. S., Dissociation of “conscious desire” (craving) from and relapse in alcohol and cocaine dependence, Ann. Clin. Psychiatry, 6, 99, 1994.
Mollenauer, S., Bryson, R., Robinson, M., Sardo, J., and Coleman, C., EtOH Self-administration in anticipation of noise stress in C57BL/6J mice, Pharmacol. Biochem. Behav., 46, 35, 1993.
Monti P. M., Rohsenow D. J., Rubonis A. V., Niaura R. S., Sirota A. D., Colby S. M., Abrams D. B. Alcohol cue reactivity: effects of detoxification and extended exposure. J Stud Alcohol 1993; 54:235-45.
Monti P M, Rohsenow D J, Rubonis A V, Niaura R S, Sirota A D, Colby S M, Abrams D B (1993) Alcohol cue reactivity: effects of detoxification and extended exposure. J Stud Alcohol 54: 235-45
Mueller M. et al. (2008) Red Clover Extract: a Putative Source for Simultaneous Treatment of Menopausal Disorders and the Metabolic Syndrome, 15 (6):1120-31.
Nash, J., Jr. and Maickel, R. P., The role of the hypothalamic-pituitary-adrenocortical axis in post-stress induced ethanol consumption by rats, Prog Neuropsychopharmacol Biol Psychiatry, 12, 653, 1988.
O'Brien C P (1997). A range of research-based pharmacotherapies for addiction. Science 278: 66-70.
O'Brien C P, McLellan A T (1996) Myths about the treatment of addiction. Lancet 347: 237-40
Panossian A, Hovhannisyan A, Mamikonyan G, Abrahamian H, Hambardzumyan E, Gabrielian E, Goukasova G, Wikman G, Wagner H (2000) Pharmacokinetic and oral bioavailability of andrographolide from Andrographis paniculata fixed combination Kan Jang in rats and human. Phytomedicine 7: 351-64
Parichatikanond W, Suthisisang C, Dhepakson P, Herunsalee A (2010) Study of anti-inflammatory activities of the pure compounds from Andrographis paniculata (burm.f.) Nees and their effects on gene expression. Int Immunopharmacol 10: 1361-73
Pomerleau, O. F., Fertig, J., Baker, L., and Cooney, N., Reactivity to alcohol cues in alcoholics and non-alcoholics: Implications for the stimulus control analysis of drinking, Addict. Behav., 8, 1, 1983.
Pucak M L, Kaplin A I (2005) Unkind cytokines: current evidence for the potential role of cytokines in immune-mediated depression. Int Rev Psychiatry 17: 477-83
Qin L, He J, Hanes R N, Pluzarev O, Hong J S, Crews F T (2008) Increased systemic and brain cytokine production and neuroinflammation by endotoxin following ethanol treatment. J Neuroinflammation 5: 10

Qin L H, Kong L, Shi G J, Wang Z T, Ge B X (2006) Andrographolide inhibits the production of TNF-alpha and interleukin-12 in lipopolysaccharide-stimulated macrophages: role of mitogen-activated protein kinases. Biol Pharm Bull 29: 220-4
Raison C L, Capuron L, Miller A H (2006) Cytokines sing the blues: inflammation and the pathogenesis of depression. Trends Immunol 27: 24-31
Ramsey, N. F. and Van Ree, M., Emotional but not physical stress enhances intravenous cocaine self-administration in drug naive rats, Brain Res., 608, 216, 1993.
Reddy M V, Reddy M K, Gunasekar D, Caux C, Bodo B (2003) A flavanone and a dihydrodibenzoxepin from Bauhinia variegata. Phytochemistry 64: 879-82
Reddy V L, Reddy S M, Ravikanth V, Krishnaiah P, Goud T V, Rao T P, Ram T S, Gonnade R G, Bhadbhade M, Venkateswarlu Y (2005) A new bis-andrographolide ether from Andrographis paniculata nees and evaluation of anti-HIV activity. Nat Prod Res 19: 223-30
Reyes-Balaguer J, Solaz-Moreno E, Morata-Aldea C, Elorza-Montesinos P (2005) Spontaneous diabetic myonecrosis. Diabetes Care 28: 980-1
Ricote M, Li A C, Willson T M, Kelly C J, Glass C K (1998) The peroxisome proliferator-activated receptor-gamma is a negative regulator of macrophage activation. Nature 391: 79-82
Rozema E. et al. (2012) Selected Extracts of Chinese Herbal Medicines: Their Effect on NF-κB, PPARα and PPARγ and the Respective Bioactive Compounds, Evidence-Based Complementary and Alternative Medicine, Volume 2012, Article ID 983023, 10 pps.
Rostene W, Kitabgi P, Parsadaniantz S M (2007) Chemokines a new class of neuromodulator? Nat Rev Neurosci 8: 895-903
Shaham Y and Stewart J. Stress reinstates heroin-seeking in drug-free animals: an effect mimicking heroin, not withdrawal. Psychopharmacology 1995; 119:334.
Shaham, Y., Immobilization stress-induced oral opioid self-administration and withdrawal in rats: role of conditioning factors and the effect of stress on “relapse” to opioid drugs, Psychopharmacology, 111, 477, 1993.
Shen Y C, Chen C F, Chiou W F (2002) Andrographolide prevents oxygen radical production by human neutrophils: possible mechanism(s) involved in its anti-inflammatory effect. Br J Pharmacol 135: 399-406
Shi M D, Lin H H, Lee Y C, Chao J K, Lin R A, Chen J H (2008) Inhibition of cell-cycle progression in human colorectal carcinoma Lovo cells by andrographolide. Chem Biol Interact 174: 201-10
Shi P, Raizada M K, Sumners C (2010) Brain cytokines as neuromodulators in cardiovascular control. Clin Exp Pharmacol Physiol 37: e52-7
Siegal, H. A., R. G. Carlson, et al. (2003). Probable relationship between opioid abuse and heroin use. Am Fam Physician 67(5): 942, 945.
Stopponi S, Somaini L, Cippitelli A, Cannella N, Braconi S, Kallupi M, Ruggeri B, Heilig M, Demopulos G, Gaitanaris G, Massi M, Ciccocioppo R (2010) Activation of nuclear PPARgamma receptors by the antidiabetic agent pioglitazone suppresses alcohol drinking and relapse to alcohol seeking Biol Psychiatry 69: 642-9
Stormark, K. M., Laberg, J. C., Bjerland, T., Nordby, H., and Hugdahl, K., Autonomic cued reactivity in alcoholics: The effect of olfactory stimuli, Addict. Behav., 20, 571, 1995.
Substance Abuse and Mental Health Services Administration (2003). Substance Abuse and Mental Health Services Administration, Emergency Department Trends From the Drug Abuse Warning Network, Final Estimates 1995-2002, Substance Abuse and Mental Health Services Administration, Rockville, Md. Office of Applied Statistics.
Substance Abuse and Mental Health Services Administration (2004). Substance Abuse and Mental Health Services Administration, Mortality Data from the Drug Abuse Warning Network, 2002, Substance Abuse and Mental Health Services Administration, Rockville, Md. Office of Applied Statistics.
Suebsasana S, Pongnaratorn P, Sattayasai J, Arkaravichien T, Tiamkao S, Aromdee C (2009) Analgesic, antipyretic, anti-inflammatory and toxic effects of andrographolide derivatives in experimental animals. Arch Pharm Res 32: 1191-200
Swiergiel, A. H., Takahashi, L. K., and Kalin, N. H., Attenuation of stress-induced behaviour by antagonism of corticotropin-releasing factor receptors in the central amygdala in the rat, Brain Res, 623, 229, 1993.
Szabo G, Dolganiuc A, Dai Q, Pruett S B (2007) TLR4, ethanol, and lipid rafts: a new mechanism of ethanol action with implications for other receptor-mediated effects. J Immunol 178: 1243-9
Thisoda P, Rangkadilok N, Pholphana N, Worasuttayangkurn L, Ruchirawat S, Satayavivad J (2006) Inhibitory effect of Andrographis paniculata extract and its active diterpenoids on platelet aggregation. Eur J Pharmacol 553: 39-45
Tiffany, S. T. and Carter, B. L., Is craving the source of compulsive drug use?, J Psychopharmacol, 12, 23, 1998.
Tsai R Y, Jang F L, Tai Y H, Lin S L, Shen C H, Wong C S (2008) Ultra-low-dose naloxone restores the antinociceptive effect of morphine and suppresses spinal neuroinflammation in PTX-treated rats. Neuropsychopharmacology 33: 2772-82
Uddin M, Aiello A E, Wildman D E, Koenen K C, Pawelec G, de Los Santos R, Goldmann E, Galea S Epigenetic and immune function profiles associated with posttraumatic stress disorder. Proc Natl Acad Sci USA 107: 9470-5
Umhau J C, Schwandt M L, Usala J, Geyer C, Singley E, George D T, Heilig M (2011) Pharmacologically induced alcohol craving in treatment seeking alcoholics correlates with alcoholism severity, but is insensitive to acamprosate. Neuropsychopharmacology 36: 1178-86
Volpicelli J. R., Alternan A. I., Hayashida M., O'Brien C. P. Naltrexone in the treatment of alcohol dependence. Arch Gen Psychiatry 1992; 49:876-880.
Wallace, B. C., Psychological and environmental determinants of relapse in crack cocaine smokers, J Subst Abuse Treat, 6, 95, 1989.
Wang, H M et al. (2010), PPARgamma Agonist Curcumin Reducs the Amyloid-Beta-Stimulated Responses in Primary Astrocytes, J. Alzheimers Dis. 20(4):1189-99.
Wang T, Liu B, Zhang W, Wilson B, Hong J S (2004) Andrographolide reduces inflammation-mediated dopaminergic neurodegeneration in mesencephalic neuron-glia cultures by inhibiting microglial activation. J Pharmacol Exp Ther 308: 975-83
Wang Y J, Wang J T, Fan Q X, Geng J G (2007) Andrographolide inhibits NF-kappaBeta activation and attenuates neointimal hyperplasia in arterial restenosis. Cell Res 17: 933-41
Weiss, F. and Ciccocioppo, R., Environmental stimuli potently reinstate alcohol-seeking behaviour: Effect of repeated alcohol intoxication, Soc. Neurosci. Abstr., 25, 1081, 1999.
Weiss, F., Maldonado-Vlaar, C. S., Parsons, L. H., Kerr, T. M., Smith, D. L., and Ben-Shahar, O., Control of cocaine-seeking behaviour by drug-associated stimuli in rats: Effects on recovery of extinguished operant responding and extracellular dopamine levels in amygdala and nucleus accumbens, Proc. Natl. Acad. Sci. USA, 97, 4321, 2000.
Wiart C, Kumar K, Yusof M Y, Hamimah H, Fauzi Z M, Sulaiman M (2005) Antiviral properties of ent-labdene diterpenes of Andrographis paniculata nees, inhibitors of herpes simplex virus type 1. Phytother Res 19: 1069-70

Xia Y F, Ye B Q, Li Y D, Wang J G, He X J, Lin X, Yao X, Ma D, Slungaard A, Hebbel R P, Key N S, Geng J G (2004) Andrographolide attenuates inflammation by inhibition of NF-kappa B activation through covalent modification of reduced cysteine 62 of p50. J Immunol 173: 4207-17
Xu Y, Chen A, Fry S, Barrow R A, Marshall R L, Mukkur T K (2007) Modulation of immune response in mice immunised with an inactivated Salmonella vaccine and gavaged with Andrographis paniculata extract or andrographolide. Int Immunopharmacol 7: 515-23
Yan Y, Yamada K, Niwa M, Nagai T, Nitta A, Nabeshima T (2007) Enduring vulnerability to reinstatement of methamphetamine-seeking behavior in glial-cell-line-derived neurotrophic factor mutant mice. FASEB J 21: 1994-2004
Yu B C, Chang C K, Su C F, Cheng J T (2008) Mediation of beta-endorphin in andrographolide-induced plasma glucose-lowering action in type I diabetes-like animals. Naunyn Schmiedebergs Arch Pharmacol 377: 529-40
Yu X, Shao X. G., Sun H., Li Y. N., Yang J., Deng Y. C., Huang Y. G. Activation of cerebral peroxisome proliferator-activated receptors gamma exerts neuroprotection by inhibiting oxidative stress following pilocarpine-induced status epilepticus. Brain Res. 2008; 1200C:146-58.
Zalcman S, Savina I, Wise R A (1999) Interleukin-6 increases sensitivity to the locomotor-stimulating effects of amphetamine in rats. Brain Res 847: 276-83
Zalcman S., Savina I., Wise R. A. (1999). Interleukin-6 increases sensitivity to the locomotor-stimulating effects of amphetamine in rats. Brain Res 847: 276-283.

1. A method of treating substance addiction or an impulse control disorder or reducing the likelihood of relapse use of an addictive substance or practice of a behavior associated with an impulse control disorder, comprising administering to a subject in need thereof a composition comprising an effective amount of Andrographis paniculata or an active substance or extract of Andrographis paniculata, wherein the composition is effective to treat the substance addiction or impulse control disorder or reduce the likelihood of relapse use of the addictive substance or practice of the behavior associated with the impulse control disorder.

2. The method of claim 1, wherein the composition comprises an extract of Andrographis paniculata in a solvent.

3. The method of claim 1, wherein the composition comprises dehydroandrographolide, andrographolide or neoandrographolide.

4. The method of claim 3, wherein the composition comprises andrographolide.

5. The method of claim 1, where in the composition further comprises at least one polyunsaturated fatty acid that upregulates PPARγ.

6. The method of claim 5, wherein the composition comprises a polyunsaturated fatty acid selected from eicosapentaenoic acid, conjugated linoleic acid and docosahexaenoic acid.

7. The method of claim 1, wherein the composition further comprises an effective amount of at least one botanical that upregulates PPARγ selected from rose oil, citronellol and/or geraniol, pomegranant seed oil, abscisic acid, punicic acid, red clover extract, genistein, Biochanin A, curcumin, Cornus kousa, Cistus Salvifolius, Glycyrrhiza glabra roots, Albizia julibrissin, Arisaema sp., Cnidium monnieri, Pinellia ternata and Tribulus terrestris, or an active substance or extract thereof.

8. The method of claim 1, wherein the addictive substance is selected from alcohol, nicotine, marijuana, a marijuana derivative, an opioid agonist, a benzodiazepine, a barbiturate, or a psychostimulant.

9. The method of claim 1, wherein the impulse control disorder is selected from pathological gambling, pathological overeating, pathological use of electronic devices, pathological use of electronic video games, pathological use of electronic communication devices, pathological use of cellular telephones, addiction to pornography, sex addiction, obsessive compulsive disorder, compulsive spending, binge eating disorder, food addiction, anorexia, bulimia, intermittent explosive disorder, kleptomania, pyromania, trichotillomania, compulsive overexercising, and compulsive overworking
10. A composition for use in treating substance addiction or an impulse control disorder or reducing the likelihood of relapse use of an addictive substance or practice of a behavior associated with an impulse control disorder, comprising an effective amount of Andrographis paniculata or an active substance or extract of Andrographis paniculata in a nutraceutical base, wherein the composition is effective to treat the substance addiction or impulse control disorder or reduce the likelihood of relapse use of the addictive substance or practice of the behavior associated with the impulse control disorder.

11. The composition of claim 10, wherein the composition comprises an extract of Andrographis paniculata in a solvent.

12. The composition of claim 10, wherein the composition comprises dehydroandrographolide, andrographolide or neoandrographolide.

13. The composition of claim 12, wherein the composition comprises andrographolide.

14. The composition of claim 10, where in the composition further comprises at least one polyunsaturated fatty acid that upregulates PPARγ.

15. The composition of claim 14, wherein the composition comprises a polyunsaturated fatty acid selected from polyunsaturated fatty acid selected from eicosapentaenoic acid, conjugated linoleic acid and docosahexaenoic acid.

16. The composition of claim 10, wherein the composition further comprises an effective amount of at least one botanical that upregulates PPARγ selected from rose oil, citronellol and/or geraniol, pomegranant seed oil, abscisic acid, punicic acid, red clover extract, genistein, Biochanin A, curcumin, Cornus kousa, Cistus Salvifolius, Glycyrrhiza glabra roots, Albizia julibrissin, Arisaema sp Cnidium monnieri, Pinellia ternata and Tribulus terrestris, or an active substance or extract thereof.

17. A method of treating substance addiction or an impulse control disorder or reducing the likelihood of relapse use of an addictive substance or practice of a behavior associated with an impulse control disorder, comprising administering to a subject in need thereof a composition comprising an effective amount of at least one botanical that upregulates PPARγ selected from Andrographis paniculata rose oil, citronellol and/or geraniol, pomegranant seed oil, abscisic acid, punicic acid, red clover extract, genistein, Biochanin A, curcumin, Cornus kousa, Cistus Salvifolius, Glycyrrhiza glabra roots, Albizia julibrissin, Arisaema sp., Cnidium monnieri, Pinellia ternata and Tribulus terrestris, or an active substance or extract thereof, wherein the composition is effective to treat the substance addiction or impulse control disorder or reduce the likelihood of relapse use of the addictive substance or practice of the behavior associated with the impulse control disorder.

18. A composition for use in treating substance addiction or an impulse control disorder or reducing the likelihood of relapse use of an addictive substance or practice of a behavior associated with an impulse control disorder, comprising an effective amount of at least one botanical that upregulates PPARγ selected from Andrographis paniculata rose oil, citronellol and/or geraniol, pomegranant seed oil, abscisic acid, punicic acid, red clover extract, genistein, Biochanin A, curcumin, Cornus kousa, Cistus Salvifolius, Glycyrrhiza glabra roots, Albizia julibrissin, Arisaema sp., Cnidium monnieri, Pinellia ternata and Tribulus terrestris, or an active substance or extract thereof, in a nutraceutical base, wherein the composition is effective to treat the substance addiction or impulse control disorder or reduce the likelihood of relapse use of the addictive substance or practice of the behavior associated with the impulse control disorder.
